(19) United States
(12) Patent Application Publication
(10) Pub. No.: US 2010/0104605 A1
(43) Pub. Date: Apr. 29, 2010

(54) Method for preventing and treating influenza

(75) Inventors: Chee-Keung Chung, Hong Kong (CN); Li Zuo, Hong Kong (CN)

Correspondence Address: ANDREWS KURTH LLP, 1350 I STREET, N.W., SUITE 1100, WASHINGTON, DC, 20005, US

(73) Assignee: Kindway International Limited

(21) Appl. No.: 12/588,588

(22) Filed: Oct. 20, 2009

Related U.S. Application Data
(63) Continuation-in-part of application No. 11/878,644, filed on Jul. 25, 2007, now abandoned.

(51) Int. Cl. A61K 39/00; C12N 3/00; A61K 36/06

(52) U.S. Cl. 424/274.1; 435/242; 424/195.15

(57) ABSTRACT

The present invention provides a method for making pressure-induced ganoderma spores, for using the pressure-induced ganoderma spores to ameliorate and/or prevent influenza in a mammal; and to stimulating the host immune system in a mammal. The method for making the pressure-induced ganoderma spores includes (1) soaking ganoderma spores in a solution; (2) drying the soaked ganoderma spores using a freeze-drying or a vacuum-drying method; (3) breaking the sporoderms of the dried ganoderma spores with enzyme or a mechanical method; (4) pressurizing and depressurizing the sporoderm-broken ganoderma spores to obtain the pressure-induced sporoderm-broken ganoderma spores. The methods for ameliorating and/or preventing influenza, and for stimulating the host immune system include orally administering a composition comprising the pressure-induced ganoderma spores to a mammal susceptible to or suffering from influenza. The present invention also provides the pressure-induced ganoderma spores.

### RELATED APPLICATION

The present invention is a Continuation-in-part (CIP) and claims the priority of U.S. patent application Ser. No. 11/878,644, filed on Jul. 25, 2007, the context of which is herein incorporated by reference.

### FIELD OF THE INVENTION

This invention generally relates to a method for making pressure-induced ganoderma spores which has superior effect on ameliorating the symptoms associated with influenza infection. The present invention also provides the pressure-induced ganoderma spores made by the method described herein.

### BACKGROUND OF THE INVENTION

Influenza, commonly known as the flu, is a viral infection of the lungs characterized by fever, cough, and severe muscle aches. Typically, influenza is transmitted from infected mammals through the air by coughs or sneezes creating aerosols containing the virus, and from infected birds through their droppings. Influenza can also be transmitted by saliva, nasal secretions, feces and blood. Infections either occur through direct contact with these bodily fluids, or by contact with contaminated surfaces. Flu viruses remain infectious for over 30 days at 0° C. (32° F.) and about one week at human body temperature, although they are rapidly inactivated by disinfectants and detergents.

Influenza spreads around the world in seasonal epidemics, killing millions of people in pandemic years and hundreds of thousands in non-pandemic years. It was responsible for the most devastating plague in human history—the “Spanish” flu that swept around the world in 1918 killing 675,000 people in the U.S. and an estimated 20-50 million people worldwide. Influenza pandemics often result from the spread of a flu virus between animal species. Recently, a deadly avian strain of H5N1 has posed the greatest influenza pandemic threat. However, this virus has not yet mutated to spread easily between people.

Besides the rapid start of the outbreaks and the large numbers of people affected, the flu is an important disease because it can cause serious complications. Most people who get the flu get better within a week (although they may have a lingering cough and tire easily for a while longer). In the elderly and infirm, however, flu is a major cause of disability and death, often as a result of secondary infection of the lungs by bacteria.

Vaccinations against influenza are most common in high-risk humans in industrialized countries and farmed poultry. The most common human vaccine is the trivalent flu vaccine that contains purified and inactivated material from three viral strains. Typically this vaccine includes material from two influenza A virus subtypes and one influenza B virus strain. A vaccine formulated for one year may be ineffective in the following year, since the influenza virus changes every year and different strains become dominant. Antiviral drugs can be used to treat influenza, with neuraminidase inhibitors being particularly effective. The antiviral drugs, however, are expensive and have serious side effects.

Therefore, there still exist a need for a safe and effective way to prevent or treat influenza at a low cost.

### SUMMARY OF THE INVENTION

The present invention provides a method for making pressure-induced sporoderm-broken ganoderma spores (i.e., sporoderm-broken ganoderma spores wherein the level of bioactivity of the ganoderma spores is induced by pressure). The method includes (1) soaking ganoderma spores in a solution; (2) drying the soaked ganoderma spores using a freeze-drying or a vacuum-drying method; (3) breaking the sporoderm of the dried ganoderma spores with enzyme or a mechanical method; (4) pressurizing and depressurizing the sporoderm-broken ganoderma spores to obtain the pressure-induced sporoderm-broken ganoderma spores.

The ganoderma spores are preferred to be the spores of Ganoderma lucidum.

The mature ganoderma spores should be selected for making the pressure-induced sporoderm-broken ganoderma spores.

The preferred solution for soaking the ganoderma spores is preferably water.

The preferred drying method is a freeze-drying method.

The preferred method for breaking the sporoderm of the ganoderma spores is by using a mechanical means.

The pressurizing and depressurizing process is preferred to be conducted in a pressure chamber at a pressure of about 1 to 30 M Pa, more favorably at about 5-10 M Pa.

It is preferred to sterilized the pressure-induced sporoderm-broken ganoderma spores before packing them into a capsule.

The present invention also provides a method for ameliorating the systems associated with influenza infection in a mammal. The method comprises administering to a mammal susceptible to or suffering from influenza an effective amount of the pressure-induced sporoderm-broken Ganoderma lucidum spores. The influenza virus is preferably an influenza A virus, particularly the FM1 strain influenza A virus.

The effective amount of the pressure-induced sporoderm-broken Ganoderma lucidum spores. to be used for preventing influenza or ameliorating symptoms of influenza is between 0.01 and 20 mg/kg body weight/day.

To prevent the influenza infection, the pressure-induced sporoderm-broken ganoderma spores is preferably to be given orally to the mammal consecutively period of at least seven days.

The present invention further provides a method for stimulating the host immune system in a mammal susceptible to or suffering from influenza. The method comprises administering to the mammal a composition comprising an effective amount of the pressure-induced sporoderm-broken ganoderma spores. The influenza virus is preferably an influenza A virus, particularly the FM1 strain influenza A virus.

The effective amount of the pressure-induced sporoderm-broken ganoderma spores to be used for stimulating the host immune system is between 0.01 and 20 mg/kg body weight/day. Preferably, the pressure-induced sporoderm-broken ganoderma spores are administered orally for a period of at least seven days.

Finally, the present invention provides the pressure-induced sporoderm-broken ganoderma spores, which is prepared by (1) soaking the ganoderma spores in a solution; (2) drying the soaked ganoderma spores using a freeze-drying or a vacuum-drying method; (3) breaking the sporoderm of the dried ganoderma spores with enzyme or a mechanical method; and (4) pressurizing and depressurizing the sporoderm-broken ganoderma spores to obtain the pressure-induced sporoderm-broken ganoderma spores.

### BRIEF DESCRIPTION OF DRAWINGS

The patent application file contains at least one drawing executed in color. Copies of this patent application publication with color drawing(s) will be provided by the office upon request and payment of the necessary fee.

FIG. 1 is a diagram showing comparison of the thymus indexes of mice in different groups 5 days post inoculation of influenza virus. 1: Germination-activated sporoderm-broken ganoderma spores (GA-GS) high dosage group (1.2 g/Kg/d); 2: GA-GS medium dosage group (0.6 g/Kg/d); 3: GA-GS low dosage group (0.3 g/Kg/d); 4: CMC control group (0.5% carboxymethyl cellulose in double distilled water); 5: normal control group (0.5 ml distilled water); 6: virus control group (0.5 ml of 1/200 FM1).

FIG. 2 is a diagram showing comparison of spleen indexes of mice in different groups 5 days post inoculation of influenza virus. 1: GA-GS high dosage group (1.2 g/Kg/d); 2: GA-GS medium dosage group (0.6 g/Kg/d); 3: GA-GS low dosage group (0.3 g/Kg/d); 4: CMC control group (0.5% carboxymethyl cellulose in double distilled water); 5: normal control group (0.5 ml distilled water); 6: virus control group (0.5 ml of 1/200 FM1).

FIG. 3 is a diagram showing comparison of lung indexes of mice in different groups 5 days post inoculation of influenza virus (mg/g). 1: GA-GS high dosage group (1.2 g/Kg/d); 2: GA-GS medium dosage group (0.6 g/Kg/d); 3: GA-GS low dosage group (0.3 g/Kg/d); 4: CMC control group (0.5% carboxymethyl cellulose in double distilled water); 5: normal control group (0.5 ml distilled water); 6: virus control group (0.5 ml of 1/200 FM1).

FIG. 4 is a composite of diagrams showing flow cytometry analysis of CD19⁺ B cells in the peripheral blood of the experimental mice.

FIG. 5 is a diagram showing the effects of GA-GS on CD19⁺ cell counts in the peripheral blood of the experimental mice. 1: GA-GS high dosage group (1.2 g/Kg/d); 2: GA-GS medium dosage group (0.6 g/Kg/d); 3: GA-GS low dosage group (0.3 g/Kg/d); 4: CMC control group (0.5% carboxymethyl cellulose in double distilled water); 5: normal control group (0.5 ml distilled water); 6: virus control group (0.5 ml of 1/200 FM1).

FIG. 6 is a composite of diagrams showing flow cytometry analysis of CD4⁺ T cells in the peripheral blood of the experimental mice.

FIG. 7 is a diagram showing the effects of GA-GS on CD4⁺ T cell counts in the peripheral blood of the experimental mice. 1: GA-GS high dosage group (1.2 g/Kg/d); 2: GA-GS medium dosage group (0.6 g/Kg/d); 3: GA-GS low dosage group (0.3 g/Kg/d); 4: CMC control group (0.5% carboxymethyl cellulose in double distilled water); 5: normal control group (0.5 ml distilled water); 6: virus control group (0.5 ml of 1/200 FM1).

FIG. 8 is a composite of diagrams showing flow cytometry analysis of CD8⁺ T cells in the peripheral blood of the experimental mice.

FIG. 9 is a diagram showing the effects of GA-GS on CD8⁺ T cell counts in the peripheral blood of the experimental mice. 1: GA-GS high dosage group (1.2 g/Kg/d); 2: GA-GS medium dosage group (0.6 g/Kg/d); 3: GA-GS low dosage group (0.3 g/Kg/d); 4: CMC control group (0.5% carboxymethyl cellulose in double distilled water); 5: normal control group (0.5 ml distilled water); 6: virus control group (0.5 ml of 1/200 FM1).

FIG. 10 is a composite of diagrams showing flow cytometry analysis of NK cells in the peripheral blood of the experimental mice.

FIG. 11 is a diagram showing effects of GA-GS on CD49b⁺ NK cell counts in the peripheral blood of the experimental mice. 1: GA-GS high dosage group (1.2 g/Kg/d); 2: GA-GS medium dosage group (0.6 g/Kg/d); 3: GA-GS low dosage group (0.3 g/Kg/d); 4: CMC control group (0.5% carboxymethyl cellulose in double distilled water); 5: normal control group (0.5 ml distilled water); 6: virus control group (0.5 ml of 1/200 FM1).

FIG. 12 is a composite of diagrams showing flow cytometry analysis of NKT cells in the peripheral blood of the experimental mice.

FIG. 13 is a diagram showing effects of GA-GS on NKT cell counts in the peripheral blood of the experimental mice. 1: GA-GS high dosage group (1.2 g/Kg/d); 2: GA-GS medium dosage group (0.6 g/Kg/d); 3: GA-GS low dosage group (0.3 g/Kg/d); 4: CMC control group (0.5% carboxymethyl cellulose in double distilled water); 5: normal control group (0.5 ml distilled water); 6: virus control group (0.5 ml of 1/200 FM1).

FIG. 14 is a standard curve for flow cytometry detection of IL-2.

FIG. 15 is a diagram showing effects of GA-GS on serum IL-2 levels in the experimental mice. 1: GA-GS high dosage group (1.2 g/Kg/d); 2: GA-GS medium dosage group (0.6 g/Kg/d); 3: GA-GS low dosage group (0.3 g/Kg/d); 4: CMC control group (0.5% carboxymethyl cellulose in double distilled water); 5: normal control group (0.5 ml distilled water); 6: virus control group (0.5 ml of 1/200 FM1).

FIG. 16 is a standard curve for flow cytometry detection of TNF-α.

FIG. 17 is a diagram showing effects of GA-GS on serum TNF-α levels in the experimental mice. 1: GA-GS high dosage group (1.2 g/Kg/d); 2: GA-GS medium dosage group (0.6 g/Kg/d); 3: GA-GS low dosage group (0.3 g/Kg/d); 4: CMC control group (0.5% carboxymethyl cellulose in double distilled water); 5: normal control group (0.5 ml distilled water); 6: virus control group (0.5 ml of 1/200 FM1).

FIG. 18 is a standard curve for flow cytometry detection of IFN-γ.

FIG. 19 is a diagram showing effects of GA-GS on serum IFN-γ levels in the experimental mice. 1: GA-GS high dosage group (1.2 g/Kg/d); 2: GA-GS medium dosage group (0.6 g/Kg/d); 3: GA-GS low dosage group (0.3 g/Kg/d); 4: CMC control group (0.5% carboxymethyl cellulose in double distilled water); 5: normal control group (0.5 ml distilled water); 6: virus control group (0.5 ml of 1/200 FM1).

FIG. 20 is a standard curve for flow cytometry detection of IL-4.

FIG. 21 is a diagram showing the effects of GA-GS on serum IL-4 levels in the experimental mice. 1: GA-GS high dosage group (1.2 g/Kg/d); 2: GA-GS medium dosage group (0.6 g/Kg/d); 3: GA-GS low dosage group (0.3 g/Kg/d); 4: CMC control group (0.5% carboxymethyl cellulose in double distilled water); 5: normal control group (0.5 ml distilled water); 6: virus control group (0.5 ml of 1/200 FM1).

FIG. 22 is a standard curve plot for flow cytometry detection of IL-5.

FIG. 23 is a diagram showing the effects of GA-GS on serum IL-5 levels in the experimental mice. 1: GA-GS high dosage group (1.2 g/Kg/d); 2: GA-GS medium dosage group (0.6 g/Kg/d); 3: GA-GS low dosage group (0.3 g/Kg/d); 4: CMC control group (0.5% carboxymethyl cellulose in double distilled water); 5: normal control group (0.5 ml distilled water); 6: virus control group (0.5 ml of 1/200 FM1).

FIG. 24 is a diagram showing the immunofluorescence detection of influenza virus in the frozen section of mouse lung and trachea.

FIG. 25 is a composite of pictures showing detection of influenza virus particles in the trachea of experimental mice by immunofluorescence. Panel A: P group, positive, ++++, panel B: L group, positive, +++, panel C: H group, positive, ++; panel D: N group, negative.

FIG. 26 is a composite of pictures showing detection of influenza virus particles in the lung tissue of experimental mice by immunofluorescence. Panel A: H group, positive, ++; panel B: L group, positive, +++; panel C: P group, positive, ++++; panel D: N group, negative.

FIG. 27 is a diagram showing the titer of hemagglutination inhibition antibody against influenza virus in mice from different experimental groups.

FIG. 28 is a composite of pictures showing HE staining of tissue sections from mice in the normal feeding control group (N group, 200×). Panel A: lung; panel B: trachea; panel C: thymus; panel D: spleen; panel E: lymph nodes.

FIG. 29 is a composite of pictures showing HE staining of lung tissue (panel A) and trachea (panel B) of mice in the GA-GS low dose group (L group, 200×). ( [symbol] indicates the pulmonary alveolus soakage in alveolar wall and bronchiole; [symbol] indicates the lung tissue thickened, no inflammation exudates was found in alveolar cavity)

FIG. 30 is a composite of pictures showing HE staining of tissues from mice in the GA-GS medium dose group (M group, 200×). Panel A: lung tissue, [symbol] indicates the lung tissue with consolidation, [symbol] indicates that pulmonary alveolus was full of inflammation exudates predominated by neutrophilic granulocyte. Panel B: trachea, [symbol] indicates the degradation, necrosis and desquamation of mucous membrane of trachea).

FIG. 31 is a composite of pictures showing HE staining of tissues from mice in the virus control group (P group, 200×). Panel A: lung tissue, [symbol] indicates the lung tissue with consolidation. [symbol] indicates that pulmonary alveolus was full of [symbol] inflammation exudates predominated neutrophilic granulocyte. Panel B: trachea, indicates the degradation, necrosis and desquamation of mucous membrane of trachea.

### DETAILED DESCRIPTION OF THE INVENTION

In describing preferred embodiments of the present invention, specific terminology is employed for the sake of clarity. In particular, two key terminologies are used throughout this application: (1) GA-GS: which stands for “germination-activated sporoderm-broken ganoderma spores. As set forth below, infra, these ganoderma spores are prepared in the following sequence: (a) a germination process; (b) an activation process; (c) a sporoderm breaking process; and (d) a drying process. (2) PI-GS: which stands for “pressure-induced sporoderm-broken ganoderma spores. As set forth below, infra, these ganoderma spores are prepared in the following sequence: (a) a germination process; (b) a freeze-drying or vacuum-drying process; (c) a sporoderm breaking process; (d) a pressurizing/depressurizing process. The GA-GS process has been described in U.S. Pat. No. 6,316,007, which is herein incorporated by reference. The PI-GS process is a new process invented by the inventor of this application. As demonstrated below, the PI-GS prepared by the PI-GS process demonstrate a much greater bioactivity and display far better anti-tumor and immunological effects, including ameliorating the symptoms of influenza, than GA-GS. Collectively, the GA-GS and PI-GS are referred to as “GS.”

The invention is not intended to be limited to the specific terminology so selected. It is to be understood that each specific element includes all technical equivalents which operate in a similar manner to accomplish a similar purpose.

One aspect of the present invention relates to a method for preventing influenza or ameliorating symptoms of influenza in a mammal susceptible to or suffering from influenza. The method comprises administering to the mammal an effective, amount of GS.

The influenza virus is an RNA virus of the family Orthomyxoviridae, which comprises the influenzaviruses, Isavirus and Thogotovirus. There are three types of influenza virus: Influenzavirus A, Influenzavirus B or Influenzavirus C. Influenza A and C infect multiple species, while influenza B almost exclusively infects humans. The type A viruses are the most virulent human pathogens among the three influenza types, and cause the most severe disease.

The influenza A virus particle or virion is 80-120 nm in diameter and usually roughly spherical, although filamentous forms can occur. Unusually for a virus, the influenza A genome is not a single piece of nucleic acid; instead, it contains eight pieces of segmented negative-sense RNA (13.5 kilobases total), which encode 11 proteins (HA, NA, NP, M1, M2, NS1, NEP, PA, PB1, PB1-F2, PB2). The best-characterised of these viral proteins are hemagglutinin and neuraminidase, two large glycoproteins found on the outside of the viral particles. Neuraminidase is an enzyme involved in the release of progeny virus from infected cells, by cleaving sugars that bind the mature viral particles. By contrast, hemagglutinin is a lectin that mediates binding of the virus to target cells and entry of the viral genome into the target cell. The hemagglutinin (HA or H) and neuraminidase (NA or N) proteins are targets for antiviral drugs. These proteins are also recognized by antibodies, i.e. they are antigens. The responses of antibodies to these proteins are used to classify the different serotypes of influenza A viruses, hence the H and N in H5N1.

The Influenza A virus can be subdivided into different serotypes based on the antibody response to these viruses. The serotypes that have been confirmed in humans, ordered by the number of known human pandemic deaths, are: H1N1, which caused “Spanish Flu”; H2N2, which caused “Asian Flu”; H3N2 which caused “Hong Kong Flu”; H5N1, which is a pandemic threat in 2006-7 flu season; H7N7, which has unusual zoonotic potential; and H1N2, which is endemic in humans and pigs.

Influenza B virus is almost exclusively a human pathogen, and is less common than influenza A. The only other animal known to be susceptible to influenza B infection is the seal. This type of influenza mutates at a rate 2-3 times lower than type A and consequently is less genetically diverse, with only one influenza B serotype. As a result of this lack of antigenic diversity, a degree of immunity to influenza B is usually acquired at an early age. However, influenza B mutates enough that lasting immunity is not possible. This reduced rate of antigenic change, combined with its limited host range (inhibiting cross species antigenic shift), ensures that pandemics of influenza B do not occur.

The influenza C virus infects humans and pigs, and can cause severe illness and local epidemics. However, influenza C is less common than the other types and usually seems to cause mild disease in children.

Another aspect of the present invention relates to a method for stimulating the host immune system in a mammal susceptible to or suffering from influenza. The method comprises administering to the mammal an effective amount of GA-GS and PI-GS.

Ganoderma spores is a brown powder that is slightly soluble in water. In U.S. Pat. No. 6,316,007, germination-activated sporoderm-broken ganoderma spores (GA-GS) are produced by the process set forth below:

I. Induction of germination: Mature and perfect spores of Ganoderma lucidum are carefully selected to undergo a soaking process to induce germination. Spores are kept in clear or distilled water, biological saline solution, or other nutritional solutions that could enable the spores of Ganoderma lucidum to germinate rapidly. Examples of nutritional solutions include coconut juice or a 1-5% malt extract solution, 0.5-25% extracts of Ganoderma lucidum sporocarps or Ganoderma lucidum capillitia, a solution containing 0.1-5% biotin, and a solution containing 0.1-3% potassium phosphate (monobasic) and magnesium sulfate. The choice of solution would depend on the soaking time required, the amount of spores to be processed and other such factors as availability of materials. One or more of the above germination solutions could be used, with the amount added being 0.1-5 times the weight of the spores of Ganoderma lucidum. The soaking time can be determined according to the temperature of the water, and usually the soaking was carried out for 30 min to 8 hours with the temperature of the water at 20-43° C. Preferably, the soaking time is 2-4 hours, and the temperature of water is 25-35° C.

II. Activation culture: The spores of Ganoderma lucidum are removed from the soaking solution and excess solution is eliminated by allowing it to drip. The spores are then placed in a well-ventilated culturing box at a constant temperature and humidity so that spore culture activation could be carried out. The relative humidity of the culture is generally set at 65-98%, the culture temperature set at 18-48° C. and the activation time may last from 30 min to 24 hours. Preferably humidity is 85-97% and temperature is 25-35° C. During activation, the cell walls of the spores of Ganoderma lucidum are clearly softened such that it is easier to penetrate the cell walls of the spores. The activation of spores of Ganoderma lucidum typically reaches a rate of more than 95%.

III. Treatment of the epispores: After the germination/activation process, the spores are treated by enzymolysis. This process is carried out at a low temperature and under conditions such that enzyme activity is maintained, using chitinase, cellulase, or other enzymes, which are commonly used in the industry. The process is complete when the epispores lost their resilience and became brittle. Alternatively, physical treatments are carried out to penetrate the cell walls, for example, micronization, roll pressing, grinding, super high pressure microstream treatment, and other mechanical methods commonly used in the industry could be carried out, with a penetration rate of over 99%.

IV. Drying or extraction: Drying is carried out at low temperature using standard methods including freeze-drying or vacuum-drying etc., which are commonly used in the industry. The obtained product has a moisture content less than 4%. After drying, the bioactive substances are extracted by water or alcohol, or by thin film condensation. The extracted bioactive substances can be further purified by dialysis to ensure no contamination in the final products. The final product can be made into purified powders, extract pastes, solutions for injection, or for oral consumption.

There has been a discovery that a different process of making the ganoderma spores leads to the production of much improved bioactive ganoderma spores. The more bioactive ganoderma spores are called the pressure-induced sporoderm-broken ganoderma spores or PI-GS. The process for making the PI-GS is described as follows:

(a) Carefully select mature and perfect Ganoderma lucidum spores (GS). Clean the GS and then soak them in clean or distilled water, biological saline solution, or other nutritional solutions, such as coconut juice or a 1-5% malt extract solution, 0.5-25% extracts of Ganoderma lucidum sporocarps or Ganoderma lucidum capillitia, a solution containing 0.1-5% biotin, and a solution containing 0.1-3% potassium phosphate (monobasic) and magnesium sulfate, for about 30 min to 8 hours with the temperature of the water at about 20-43° C. The preferred soaking solution is distilled water; the preferred soaking time is about 2-4 hours, and the preferred temperature of water is about 25-35° C.

(b) Collect the soaked GS. Dry the soaked GS at low temperature using freeze-drying or vacuum-drying. The obtained product has a moisture content less than 4%. The preferred method is freeze-drying.

(c) Break the epispores by enzymolysis or mechanical means. To break the epispores by enzyme, the treatment is carried out at a low temperature and under conditions such that enzyme activity is maintained, using chitinase, cellulase, or other enzymes, which are commonly used in the industry. The process is complete when the epispores lost their resilience and became brittle. To break the epispores by mechanical means, physical treatments are carried out to penetrate the cell walls by, for example, micronization, roll pressing, grinding, super high pressure microstream treatment, and other mechanical methods commonly used in the industry. The penetration rate is over 99% by either enzyme or mechanical treatment.

(d) Place the freeze-dried or vacuum-dried, and sporoderm-broken GS in a pressure vessel. Increase the pressure to about 1 to 30 M Pa for about 30 minutes to 24 hours. Depressurize the pressure vessel, and collect the pressured-induced GS (“PI-GS”). The preferred pressure is 5 to 10 M Pa; and the preferred time under pressure is about 1 to 3 hours.

(e) Sterilize the PI-GS before packing the PI-GS into capsules or tablets.

The ganoderma spores produced by the pressure-induced method has much improved bioactivity than the ganoderma spores produced by the gemination-activation method described in U.S. Pat. No. 6,316,007 (“the '007 patent”). Especially, when the PI-GS were used to study the tumor suppressing effect of the ganoderma spores using the same experimental design as in Experimental Example 1 of the '007 patent, the PI-GS demonstrated a 15 to 40% greater tumor suppressing rate than the GA-GS produced by the germination-activation method. Experimental Example 1 of the '007 patent studied the tumor suppressing effect of mice having reticulosarcoma (L-II). The negative control group was injected with Biological saline solutions of 20 mL/kg/d; the positive control group was orally administered with adriamycin (ADM) 1 mg/kg/d; and the low dose group was administered with 2 g of PI-GS; the medium dose group was administered with 4 g of PI-GS; and the high dose group was administered with 8 g of PI-GS. The results, as compared to Table 1 of the '007 patent, showed that each of the low dose, medium dose, and high dose groups demonstrated a 15 to 40% increased tumor suppressing rate than each of the GA-GS counterparts.

The effective amount of GA-GS or PI-GS is a dosage which is useful for preventing influenza or ameliorating influenza symptoms. Toxicity and therapeutic efficacy of GA-GS and PI-GS can be determined by standard pharmaceutical procedures in cell culture or experimental animal models, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. The data obtained from cell culture assays and animal studies can be used in formulating a range of dosages for use in humans.

Generally, appropriate dosages for administering GA-GS or PI-GS may range, for example, from about 0.01 g/kg body weight/day to about 20 g/kg body weight/day. In one embodiment, the effective amount of GA-GS or PI-GS is between 0.2 and 2 g/kg body weight/day.

Both GA-GS and PI-GS are preferred to be administered orally. The administration can be in one dose, or at intervals such as three times daily, twice daily, once daily, once every other day, or once weekly. A typical treatment regimen is one administration per day for a period of two days or longer, preferably seven days or longer. A typical prevention regimen is one administration per day during the flu season. Dosage schedules for administration of GA-GS and PI-GS can be adjusted based on the individual conditions and needs of the target. Continuous infusions may also be used after the bolus dose. The effects of any particular dosage can be monitored by suitable bioassays.

Both GA-GS and PI-GS can also be formulated into a pharmaceutical composition with a pharmaceutically acceptable carrier. As used herein the language “pharmaceutically acceptable carrier” is intended to include any and all solvents, solubilizers, fillers, stabilizers, binders, absorbents, bases, buffering agents, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary agents can also be incorporated into the compositions.

The pharmaceutical composition of the present invention is formulated to be compatible with its intended route of administration, e.g., oral or parenteral administration. Oral compositions generally include an inert diluent or an edible carrier. They can be enclosed in gelatin capsules or compressed into tablets. For the purpose of oral therapeutic administration, the active compound can be incorporated with a solid carrier and used in the form of tablets, troches, or capsules. Oral compositions can also be prepared using a fluid carrier. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Pharmaceutical compositions of GA-GS and PI-GS that are suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, such as sodium chloride, sugars, polyalcohols (e.g., manitol, sorbitol, etc.) in the composition. Prolonged absorption of the injectable compositions can be brought about by including in the composition an agent which delays absorption, for example, aluminum monostearate and gelatin.

Sterile injectable solutions can be prepared by incorporating GA-GS or PI-GS in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

It is especially advantageous to formulate oral or parenteral compositions in dosage unit form for ease of administration and uniformity of dosage. Dosage unit form as used herein includes physically discrete units suited as unitary dosages for the subject to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. The pharmaceutical compositions can be included in a container, pack, or dispenser together with instructions for administration.

The following experimental designs and result are illustrative, but not limiting the scope of the present invention. Reasonable variations, such as those occur to reasonable artisan, can be made herein without departing from the scope of the present invention. Also, in describing the invention, specific terminology is employed for the sake of clarity. However, the invention is not intended to be limited to the specific terminology so selected. It is to be understood that each specific element includes all technical equivalents which operate in a similar manner to accomplish a similar purpose.

### EXAMPLES

The following experiments evaluated the effectiveness of GA-GS and PI-GS in preventing/treating influenza in mice. Mouse models provide a useful system for evaluating influenza virus pathogenesis and immunity, and are commonly used in influenza-related studies. For example, a mouse study provided new insights into the killer flu virus that caused the pandemic in 1918 (see e.g., Kong et al., Proc Natl Acad Sci USA, 2006, 103:15987-91). Mouse models have also been used to study the pathogenesis and immunity to influenza A (H5N1) viruses isolated from humans (see e.g., Lu et al., J. Virol, 1999, 73:5903-5911), and evaluate anti-influenza virus drugs (Ison et al. J Infect Dis. 2006, 193:765-72).

### Example 1

### Materials and Methods

### (A) Materials

### Experimental Animals

Balb/c mice with a body weight of 18-22 g, half male and half female, were supplied by Shanghai Laboratory Animal Center.

Sporoderm-Broken Ganoderma lucidum Spores (Gs):

GA-GS and/or PI-GS capsule (Holistol 2036™), 0.3 g/capsule.

### Influenza Virus

Mouse-adapted influenza virus strain (FM1) is preserved in the laboratory. FM1 is an influenza A virus (serotype H1N1).

### Reagents

The TH₁/TH₂ Cytokine CBA kit used in the present study for detecting of TNF-α, IFN-γ, IL-5, IL-4 and IL-2 from mice, was purchased from Jingmei Biotech Co., Ltd. (Shenzhen, China) and was originally produced by BD PharMingen Co., Ltd (San Diego, USA). FITC-CD3, PE-CD4, PE-CD8, PE-CD49b, PE-CD19, PE-CD117 flow cytometry monoclonal antibodies were purchased from Gene Co., Ltd (Chengdu, China) and were originally produced by BioLegend (San Diego, Calif.). FITC-Goat Anti-Chicken IgG was purchased from Jingmei Biotech Co., Ltd (Shenzhen, China) and was originally produced by SouthernBiotech (USA).

### (B) Methods

### Propagation and Preservation of Mouse-Adapted Influenza Virus Strain (FM1):

Nine day old chicken embryos were inoculated with influenza virus by allantoic cavity route, and cultured for 48 hours to collect allantoic fluid. The allantoic fluid was used to infect young mice in the nasal cavity and the infected young mice were sacrificed 5 day post inoculation. The congested lungs were taken out and ground into homogenate used to inoculate 9-day old chicken embryos. The HA titer from the collected allantoic fluid was 1:2560, the samples were then aliquoted into small tubes and stored at −80° C.

### GS Dosages and Dilution:

Based on the maximum tolerant dose of GS determined in earlier experiments in Balb/c mice, the high, medium and low dosage of GS in the present experiment were 1.2 g/Kg/d, 0.6 g/Kg/d and 0.3 g/Kg/d, respectively. A solution of 0.5% carboxymethyl cellulose (CMC) in double distilled water was used as the diluent.

### Grouping of Experimental Animals:

Experimental animals were grouped and treated as described in Table 1.

TABLE 1
Animal grouping and treatment

| | group | | | | | |
|---|---|---|---|---|---|---|
| | | M | | | | |
| | H | GS | L | C | N | P |
| | GS high | medium | GS low | CMC | normal | virus |
| | dose | dose | dose | control | control | control |
| | group | group | group | group | group | group |
| mice/ | 20 | 20 | 20 | 20 | 20 | 20 |
| group | | | | | | |
| treat- | GS | GS | GS | 0.5% | distilled | — |
| ment | 1.2 g/Kg/d | 0.6 g/Kg/d | 0.3 g/Kg/d | CMC | water | |
| | (0.5 ml) | (0.5 ml) | (0.5 ml) | (0.5 ml) | (0.5 ml) | |
| inocu- | 1/200 | 1/200 | 1/200 | 1/200 | — | 1/200 |
| lation | FM1 | FM1 | FM1 | FM1 | | FM1 |
| | 0.5 ml | 0.5 ml | 0.5 ml | 0.5 ml | | 0.5 ml |

### Preparation of Chicken Anti-influenza Virus Polyclonal Antibody (Primary Antibody):

Influenza virus FM1 was inoculated to 9-day old chicken allantoic cavity. Five (5) ml of allantoic fluid was collected 72 hours post inoculation under sterile condition; the fluid was injected into the heart of a rooster uninoculated with influenza virus (no influenza virus hemagglutination inhibition antibody was detected in pre-injection blood samples). Blood was collected from the heart of the rooster 10 days post inoculation under sterile condition and serum was separated. The titer of hemagglutination inhibition antibody in the serum was detected as 1:1280, and the serum was aliquoted into a small tube and stored at −80° C.

### GS Administration and Virus Inoculation:

Balb/c mice with a body weight of 18±2 g, half male and half female, were orally administered with GS (i.e., either GA-GS or PI-GS) daily for 7 consecutive days. On the day when the administration was stopped, the mice were inoculated with the FM1 influenza virus in the nasal cavity. The animals were examined every day. Necropsy was performed on mice died more than 24 h post inoculation. All surviving mice were sacrificed five days post inoculation. The mortality and the average survival time in each treatment group were calculated.

### Post Mortem Examination of Chest and Spleen:

On day 5 post inoculation, the surviving mice were weighed. Blood samples were collected by retro-orbital bleeding. The animas were sacrificed by cervical dislocation. The lungs, thymuses and spleens were dissected and weighed with an electronic scale.

The lung, thymus and spleen indexes were calculated as follows:

thymus index=thymus weight/mice body weight;

spleen index=spleen weight/mice body weight;

lung index=lung weight/mice body weight.

Flow Cytometry Detection of CD3⁺, CD4⁺, CD8⁺, CD19⁺ and CD49b⁺ Expression in Mouse Peripheral Blood Cells:

Blood samples were obtained by retro orbital bleeding using EDTA (0.6 ml/mice) as an anticoagulant. The blood samples were mixed with an monoclonal antibody (anti-CD3, CD4, CD8, CD19, or CD49b monoclonal antibody) labeled with fluroescein and incubated in dark. After the incubation, a flow cytometry detection agent (to lyse erythrocyte) was added to the blood sample. The sample was incubated in dark at room temperature until the liquid turned clear and transparent, and washed with PBS washing buffer 3 times. The cell was resuspended with 0.2 ml PBS washing buffer by vortex and submitted to flow cytometry analysis.

### Detection of Serum Levels of TNF-α, IFN-γ, IL-5, IL-4 and IL-2 by Flow Cytometry Techniques:

Detection of serum levels of TNF-α, IFN-γ, IL-5, IL-4 and IL-2 was performed according to the operating instructions of Mouse TH₁/TH₂ Cytokine CBA kit. Briefly, a standard curve was generated using standards of mouse TNF-α, IFN-γ, IL-5, IL-4, or IL-2. Serum levels of TNF-α, IFN-γ, IL-5, IL-4 and IL-2 were then determined using corresponding standard curve.

### Preparation of Frozen Sections of Mouse Lungs and Trachea:

Fresh lung tissues were embedded in OCT embedding medium, and placed in a freezing-microtome at −18-19° C., frozen for about 1 min, and the frozen section was prepared with a thickness of 10 μm.

### Immumofluorescence Staining:

Air-dried frozen tissue sections were fixed with cold acetone for 10 min at the room temperature; washed with PBS for 3 times, incubated respectively with 5% sheep serum in PBS, then 10% bovine serum in PBS at the room temperature for 30 min to block the non-specific binding. The sections were then incubated with the primary antibody (chicken anti-influenza virus serum) at 4° C. overnight, washed with PBS, incubated with the secondary antibody (FITC-goat anti-chicken IgG) in dark at the room temperature for 1 hour, washed with PBS, and sealed with a fluorescence sealing reagent.

### Determination of the Titer of Influenza Virus Hemagglutination Inhibition Antibody:

Mouse serum was diluted by serial dilution to 1/10, 1/20, 1/40, 80, 1/160, 1/320, 1/640 and 1/1280 dilution. 0.25 ml of each dilution was added to each well, followed by 4 units of virus in a volume of 0.25 ml, and 0.5 ml 0.5/100 SRBC cell suspension buffer. Control wells contained 4, 2, 1, 0.5 and 0 units of the virus.

### The Pathological Examination of Lung, Thymus, Spleen and Lymph Node:

Mice in different groups were sacrificed by cervical dislocation. The thymus, spleen and lymph node (mesentery lymph node) were dissected, fixed in 10% formaldehyde, stained with hematoxylin & eosin (HE), and subject to the pathological examination.

### Data Analysis:

The data were analyzed statistically using the spss 11.0 software.

### Example 2

### Median Infectious Dose 50 (Id₅₀) of Adult Mice for Fm1

Seventy mice were randomly divided into 5 groups. FM1 influenza virus was diluted into four dilutions at 1/25, 1/50, 1/100 and 1/200 with a buffer. Each mouse was anaesthetized by inhalation of ether, and was individually infected with 0.1 ml virus at different dilutions in the nasal cavity. Mice in the control group received buffer only. The mice were observed for five consecutive days. Necropsy was performed on mice died after 24 hours post infection. Surviving mice were sacrificed 5 days post infection. The lungs were dissected, examined for pathological changes, and measured for the wet weights. The extent of the infection was scored as ++++, +++, ++ and +, indicating the blood congested area of lungs at 100%, 75%, 50% and 25%, respectively. The ID₅₀ for FM1 influenza virus, which is defined as the virus dose that results in infection scores of ++ and above in 50% of the infected animals, was found to be 1/200 (dilution) in the present experiment.

### Example 3

### Average Survival Time of Mice Inoculated with Influenza Virus

The average survival time of mice in different groups were calculated. As shown in Table 2, the average survival time was not statistically significant among different treatment groups. However, the mice being administered with the pressure-induced GS demonstrated a slightly better survival time (although not significant) than those being administered with the germination-activated GS.

TABLE 2
Effects of GS on the average survival time of mice.

| Group | Average survival time (days) | P |
|---|---|---|
| H | 4.9 | >0.05 |
| M | 4.9 | >0.05 |
| L | 4.8 | >0.05 |
| C | 4.8 | >0.05 |
| N | 5.0 | >0.05 |
| P | 4.7 | |

### Example 4

### Mortality of Mice Inoculated with Influenza Virus

The mortality of mice in different experimental groups were calculated. As shown in Table 3, the mortality rates were not statistically different among different groups. Similar to the findings in Example 3, the mice being administered with the PI-GS demonstrated a slightly better mortality (although not significant) than those being administered with the GA-GS.

TABLE 3
The effects of GS on mice mortality.

| Group | Mortality (%) | P |
|---|---|---|
| H | 30 | >0.05 |
| M | 10 | >0.05 |
| L | 30 | >0.05 |
| C | 20 | >0.05 |
| N | 0 | >0.05 |
| P | 30 | |

### Example 5

### Thymus, Spleen and Lung Indexes of the Experimental Mice

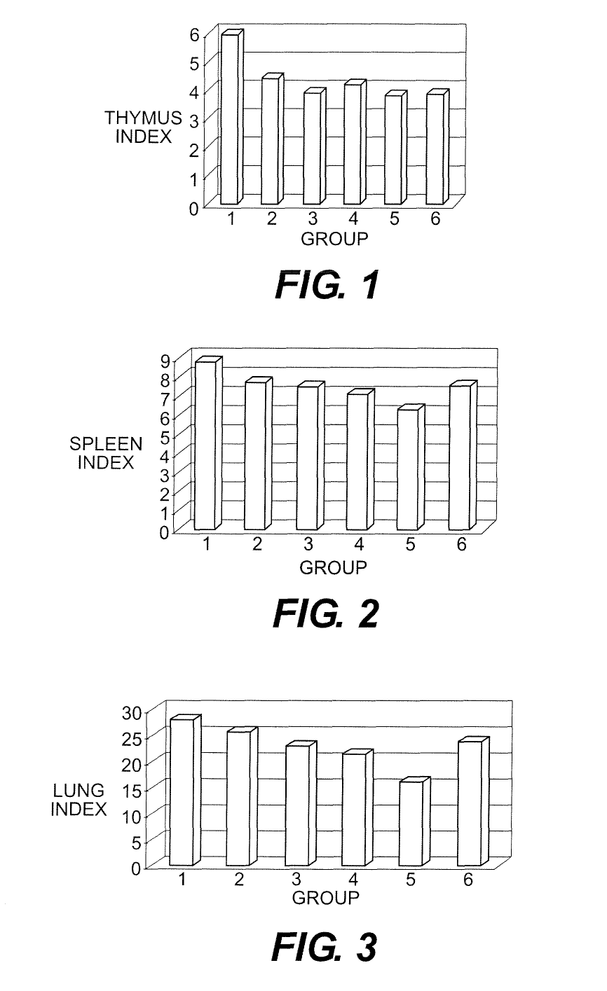

As shown in Table 4 and FIG. 1, compared with the normal control group, the thymus indexes in the GS high, medium and low dose group increased. The increase correlated with the GS dosage. The thymus index in the GS high dose group was statistically higher than that of the blank control group, CMC control group and virus control group (P<0.01). The thymus indexes of the normal control group, CMC control group and virus control group were not statistically different. Although not significantly different, the mice being administered with the PI-GS had a slightly better thumus index than those being administered with the GA-GS.

TABLE 4
Effects of GS on the thymus indexes (mg/g)

| | | Thymus index | |
|---|---|---|---|
| Group | Animals evaluated | (x ± s) | P value |
| H | 7 | 5.94 ± 2.5 | H versus M, <0.05 |
| M | 9 | 4.40 ± 0.9 | H versus L, <0.05 |
| L | 7 | 3.92 ± 1.9 | |
| C | 8 | 4.16 ± 0.8 | H versus C, <0.01 |
| N | 10 | 3.81 ± 1.0 | H versus N, <0.01 |
| P | 7 | 3.91 ± 0.3 | H versus P, <0.01 |

As shown in Table 5 and FIG. 2, compared to the normal control group, the spleen indexes of the GA-GS high, medium and low dose groups increased significantly. The increase correlated to the GS dosage and was statistically significant (P value<0.05). Specifically, spleen index of the virus control group was statistically higher than that of the normal control group (P value<0.01). Spleen index of the high dose group was statistically higher than those of the virus control group and the CMC control group (P<0.01). The spleen indexes of the PI-GS high, medium and low dose groups are at least 10% greater than that of the germination-activated GS counterparts.

TABLE 5
Effects of GS on the spleen indexes (mg/g)

| | Animals | Spleen index | |
|---|---|---|---|
| Group | evaluated | (x ± s) | P value |
| H | 7 | 8.8 ± 1.7 | |
| M | 9 | 7.7 ± 0.5 | H versus M, <0.05; M versus N, |
| | | | <0.01 |
| L | 7 | 7.4 ± 0.7 | H versus L, <0.05; L versus N, <0.05 |
| C | 8 | 7.0 ± 0.9 | H versus C, <0.01 |
| N | 10 | 6.2 ± 1.0 | H versus N, <0.01 |
| P | 7 | 7.57 ± 0.7 | H versus P, <0.05; N versus P, <0.01 |

As shown in Table 6 and FIG. 3, comparing to the normal control group, the lung indexes in the GA-GS high, medium and low dose groups increased. The increase correlated with the GS dosage. Compared with the normal control group, the increases in the GS high and medium dose groups were statistically significant (P<0.01). The lung index of the virus control group was statistically higher than that of the normal control group (P<0.01). The lung index of the CMC control group was statistically lower than that of the GS high dose group (P value<0.05). The lung indexes of the CMC control group, the GS medium and low dose groups and the virus control group were not statistically different. The lung indexes of the PI-GS high, medium, and low dose groups were significantly increased than those of the GA-GS counterparts and controls.

TABLE 6
Effects of GS on the lung indexes (mg/g)

| | Animals | | |
|---|---|---|---|
| Group | evaluated | Lung index (x ± s) | P value |
| H | 7 | 28.54 ± 7 | H versus C, <0.05 |
| M | 9 | 25.44 ± 6 | M versus N, <0.01 |
| L | 7 | 23 ± 6 | >0.05 |
| C | 8 | 21.88 ± 3 | >0.05 |
| N | 10 | 16.43 ± 7 | H versus N, <0.01 |
| P | 7 | 24.14 ± 5 | N versus P, <0.01 |

### Example 6

### Flow Cytometry Detection of CD19, CD3, CD4, CD8 and CD49b Expression in Peripheral Blood of the Experimental Mice

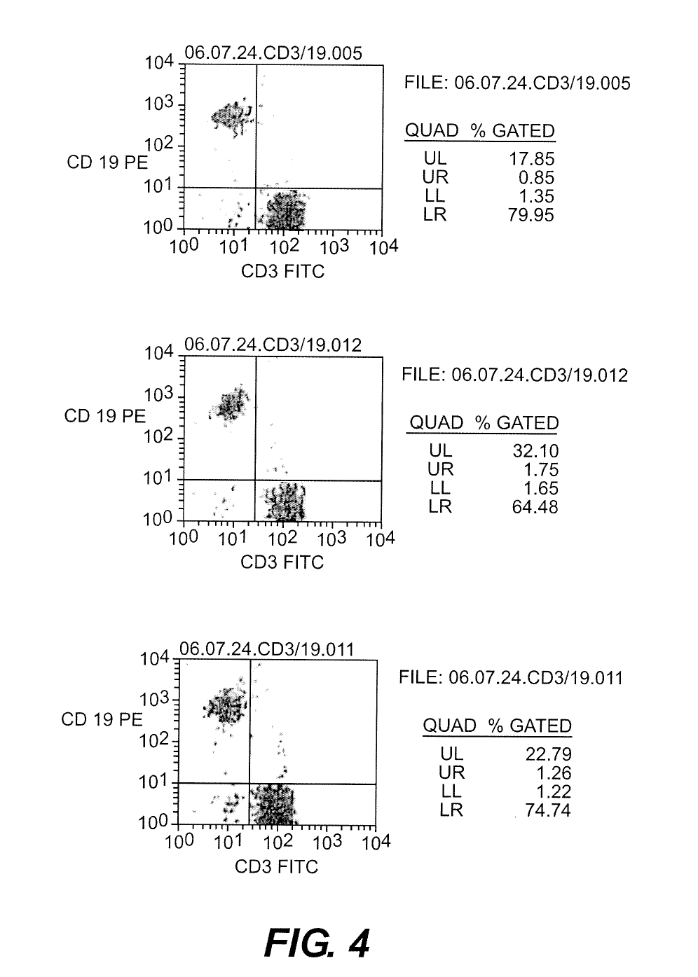

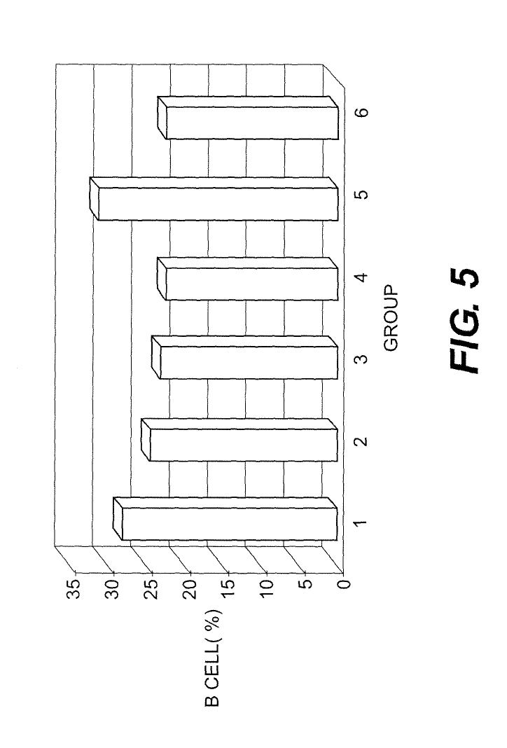

FIG. 4 shows representative flow cytometry analysis of CD19⁺ B cells in the peripheral blood of the experimental mice. As shown in Table 7 and FIG. 5, the CD19⁺B cell counts of the normal control group were higher than those of other groups. The CD19⁺ B cell counts in each GS group were higher than those of the virus control group and the CMC control group. In addition, the increase in CD19⁺ B cell counts of the GS groups correlated with the GS dosage.

TABLE 7
Effects of GS on CD19⁺ B cell counts in the
peripheral blood of the experimental mice

| Group | Animals Evaluated | CD19+ B Cell Counts (X ± s) (%) |
|---|---|---|
| H | 7 | 28.17 ± 3.43 |
| M | 9 | 24.53 ± 4.49 |
| L | 7 | 23.18 ± 3.42 |
| C | 8 | 22.63 ± 4.90 |
| N | 10 | 31.39 ± 20.12 |
| P | 7 | 22.36 ± 6.67 |

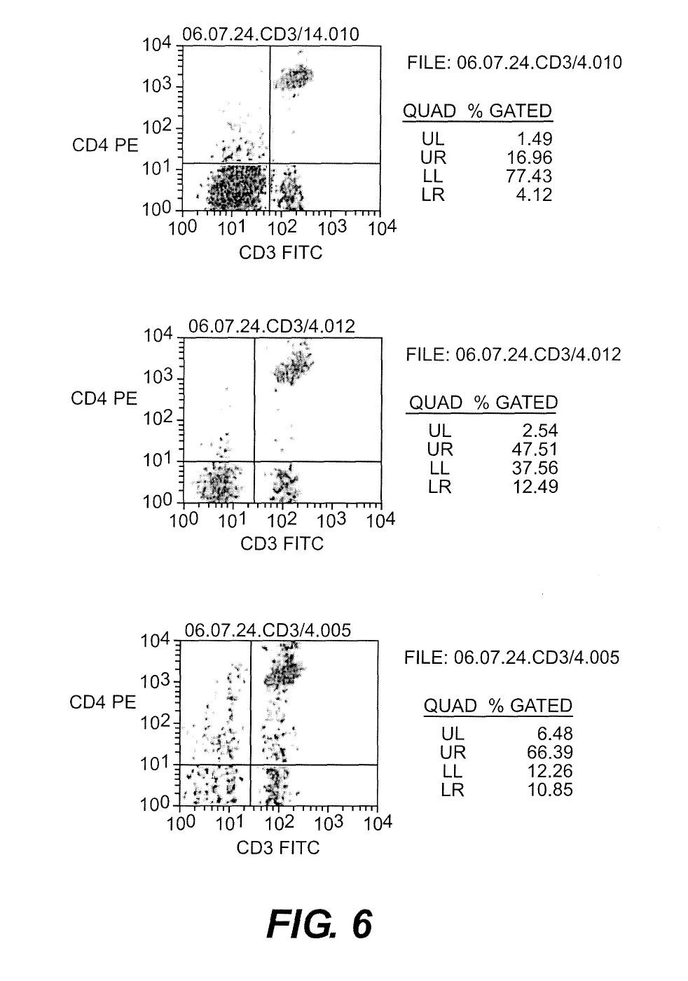

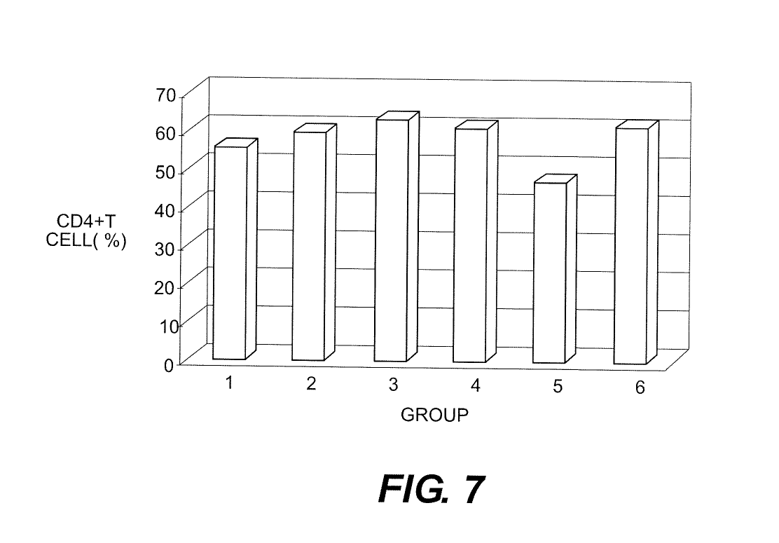

FIG. 6 shows representative flow cytometry analysis of CD4⁺ T cells in the peripheral blood of the experimental mice. As shown in Table 8 and FIG. 7, the CD4⁺ T cell counts of the germination-activated GS groups increased as the GS dosage decreased, but the changes were not statistically significant (P value>0.05). The CD4⁺ T cell counts of the GS medium dose group, the GS low dose group, the virus control group and the CMC control group were all statistically higher that those of the normal control group (P<0.05). The study using the PI-GS did not demonstrate significant difference from those of the GA-GS groups, although the data clearly demonstrated an increase in the CD4⁺ T cell counts.

TABLE 8
Effects of GS on CD4⁺ T cell counts in the peripheral
blood of experimental mice

| | Animals | CD4+ T cell | |
|---|---|---|---|
| Group | evaluated | counts (x ± s) (%) | P value |
| H | 7 | 55.81 ± 3.68 | |
| M | 9 | 59.54 ± 6.64 | M versus N, <0.05 |
| L | 7 | 63.35 ± 7.07 | L versus N, <0.01 |
| C | 8 | 61.08 ± 10.31 | C versus N, <0.01 |
| N | 10 | 47.13 ± 10.52 | |
| P | 7 | 61.72 ± 3.91 | N versus P, <0.01 |

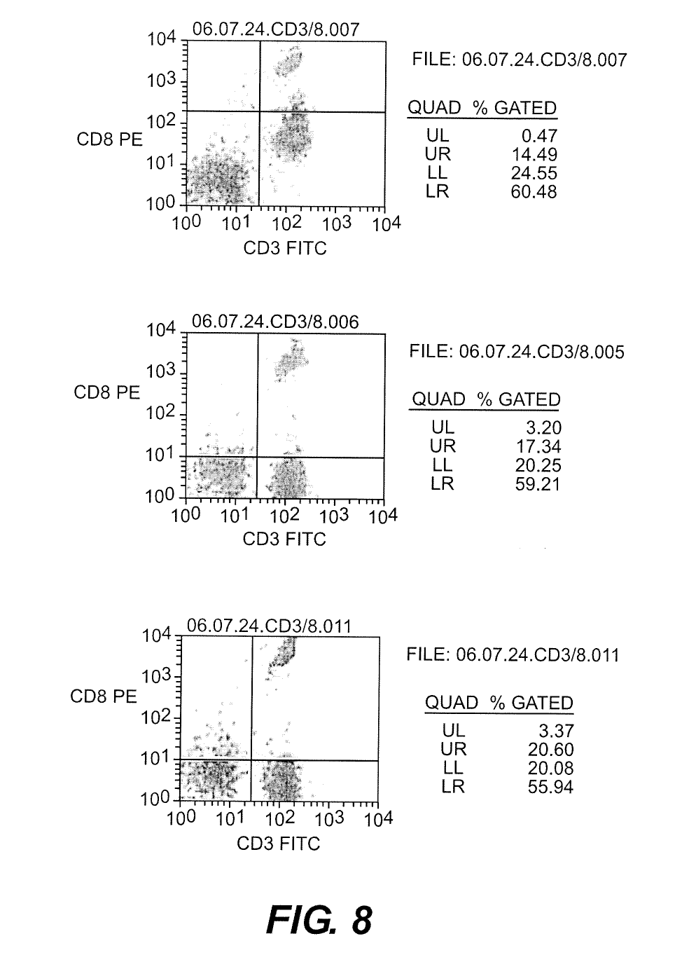

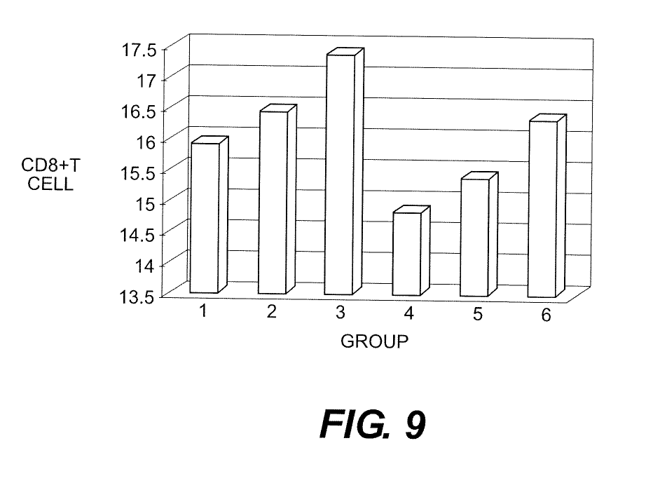

FIG. 8 shows representative flow cytometry analysis of CD8⁺ T cells in the peripheral blood of the experimental animals. As shown in Table 9 and FIG. 9, the CD8⁺ T cell counts of the GA-GS groups were higher than those of the CMC control group and the normal control group. The CD8⁺ T cell counts decreased as the GA-GS dosage increased. However, these changes were not statistically significant (P>0.05). No significant difference between the PI-GS groups and the GA-GS groups was observed.

TABLE 9
Effects of GS on CD8⁺ T cell counts in the peripheral
blood of the experimental mice

| | Animals | |
|---|---|---|
| Group | evaluated | CD8⁺ T cell counts (x ± s) (%) |
| H | 7 | 15.89 ± 2.18 |
| M | 9 | 16.42 ± 2.23 |
| L | 7 | 17.36 ± 3.43 |
| C | 8 | 14.85 ± 2.27 |
| N | 10 | 15.39 ± 4.07 |
| P | 7 | 16.35 ± 3.94 |

Note:
The statistical analysis demonstrated that the differences in CD8⁺ T cell counts among different groups were not significant (P > 0.05).

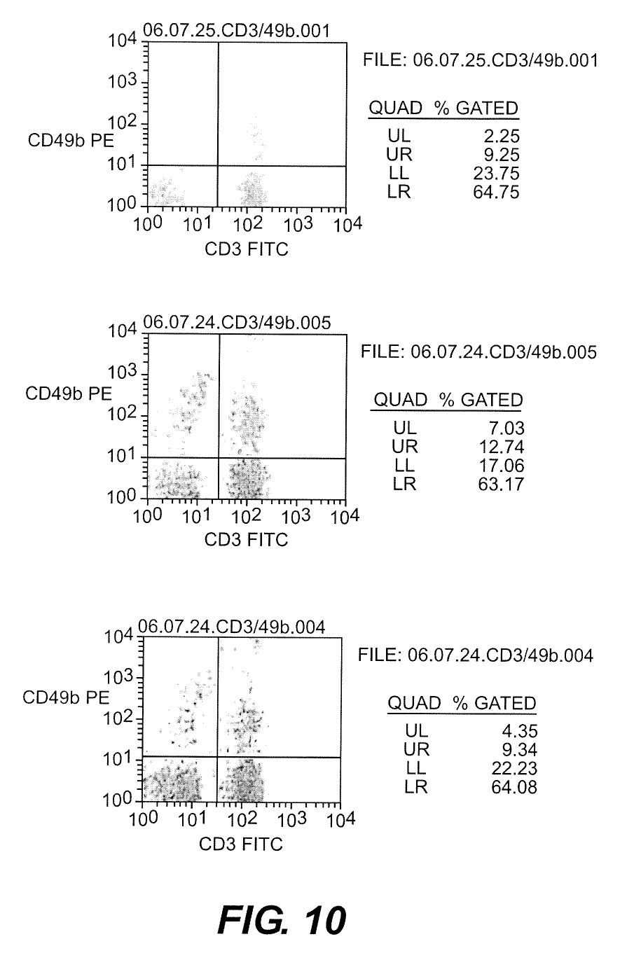

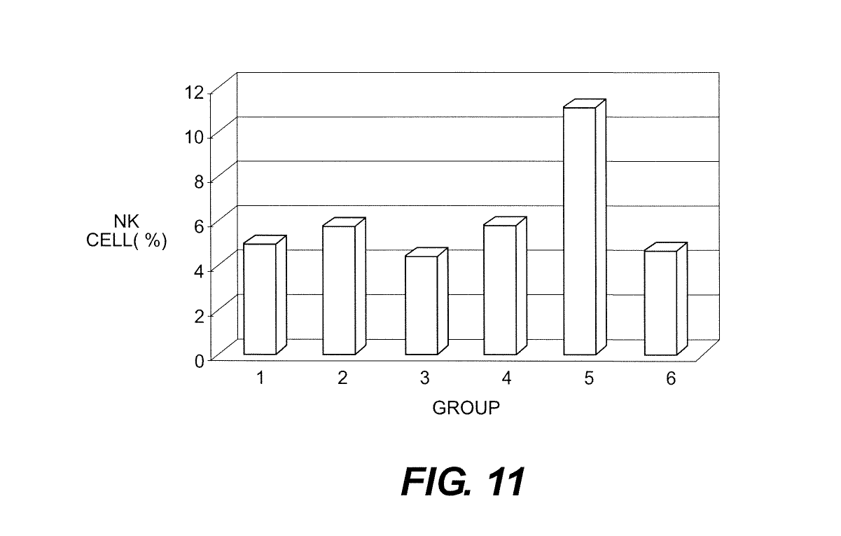

FIG. 10 shows representative flow cytometry analysis of CD49b⁺ cell counts in the peripheral blood of the experimental mice. Cells expressing CD49b but not CD3 were considered as NK cells. As shown in Table 10 and FIG. 11, the CD49b⁺ NK cell counts of the normal control group were statistically higher than those of the other groups (P<0.01).

TABLE 10
Effects of GS on CD49b⁺ NK cell counts in
the peripheral blood of the experimental mice

| | Animals | | |
|---|---|---|---|
| Group | evaluated | NK cell counts (x ± s) (%) | P value |
| H | 7 | 4.98 ± 1.17 | H versus N, <0.01 |
| M | 9 | 5.7 ± 0.98 | M versus N, <0.01 |
| L | 7 | 4.41 ± 1.37 | L versus N, <0.01 |
| C | 8 | 5.8 ± 2.95 | C versus N, <0.01 |
| N | 10 | 11.01 ± 4.56 | |
| P | 7 | 4.67 ± 0.87 | P versus N, <0.01 |

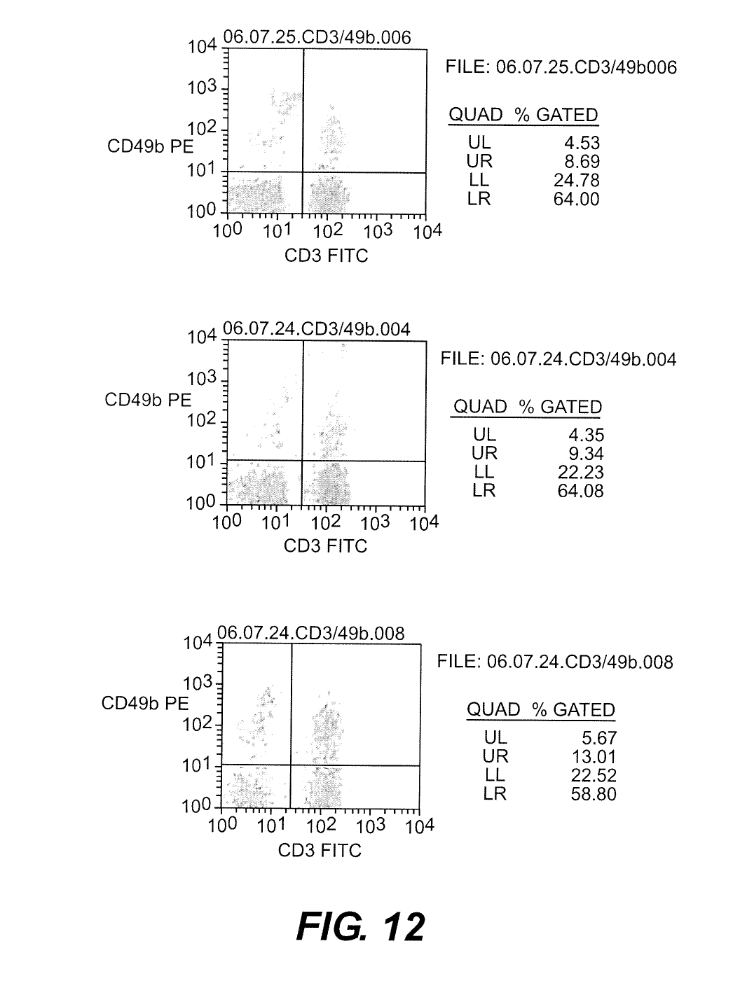

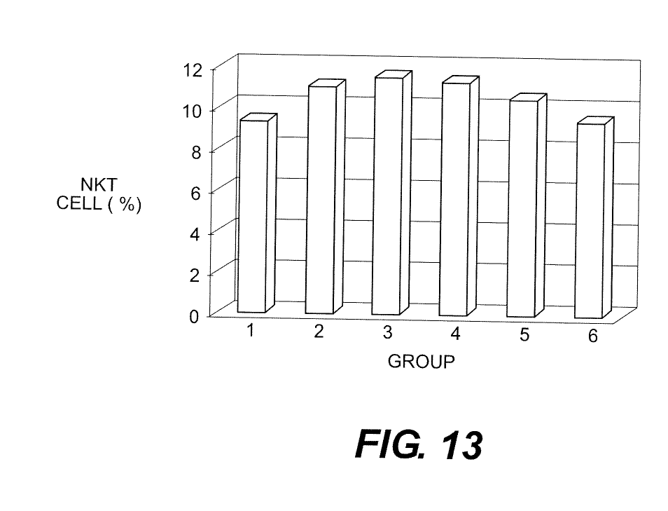

FIG. 12 shows representative flow cytometry analysis of CD49b⁺ and CD3⁺ cells in the peripheral blood of the experimental mice. Cells expresses both CD49b and CD3 were considered as NKT cell. As shown in Table 11 and FIG. 13, the NKT cell counts of the low and medium dose groups were high than that of the virus control group.

TABLE 11
Effects of GS on NKT cell counts in the peripheral
blood of the experimental mice

| | Animals | |
|---|---|---|
| Group | evaluated | NK T cell counts (x ± s) (%) |
| H | 7 | 9.40 ± 1.29 |
| M | 9 | 11.07 ± 2.23 |
| L | 7 | 11.51 ± 2.61 |
| C | 8 | 11.27 ± 4.38 |
| N | 10 | 10.47 ± 4.19 |
| P | 7 | 9.39 ± 1.61 |

### Example 7

### Flow Cytometry Analysis of IL-2, TNF-α, IFN-γ, IL-4 and IL-5 in the Blood of the Experimental Mice

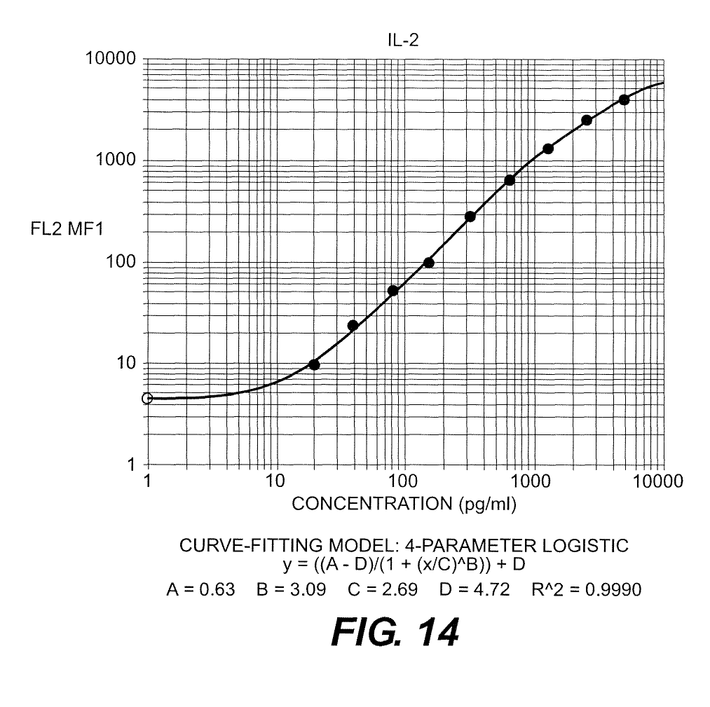

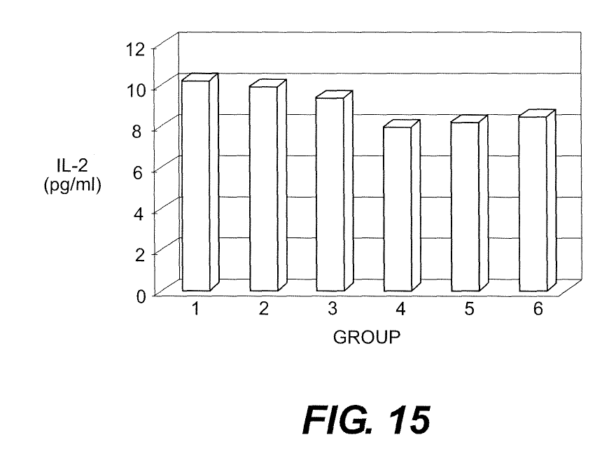

FIG. 14 shows the standard curve for flow cytometry detection of IL-2. As shown in Table 12 and FIG. 15, the serum IL-2 levels of the germination-activated GS high and medium dose groups were statistically higher than those of the CMC control group (P<0.05), while serum IL-2 levels of the other groups were not statistically different. No significant difference between the PI-GS groups and the GA-GS groups was observed.

TABLE 12
Effects of GS on serum IL-2 levels of the experimental mice

| | Animals | | |
|---|---|---|---|
| Group | evaluated | IL-2 levels (x ± s) (pg/ml) | P value |
| H | 7 | 10.25 ± 1.65 | H versus C, <0.01 |
| M | 9 | 9.98 ± 2.3 | M versus C, <0.01 |
| L | 7 | 9.46 ± 1.79 | |
| C | 8 | 8.02 ± 2.72 | |
| N | 10 | 8.38 ± 1.26 | |
| P | 7 | 8.56 ± 0.79 | |

Note:
The differences between H, M and C group were statistically significant (P < 0.05).

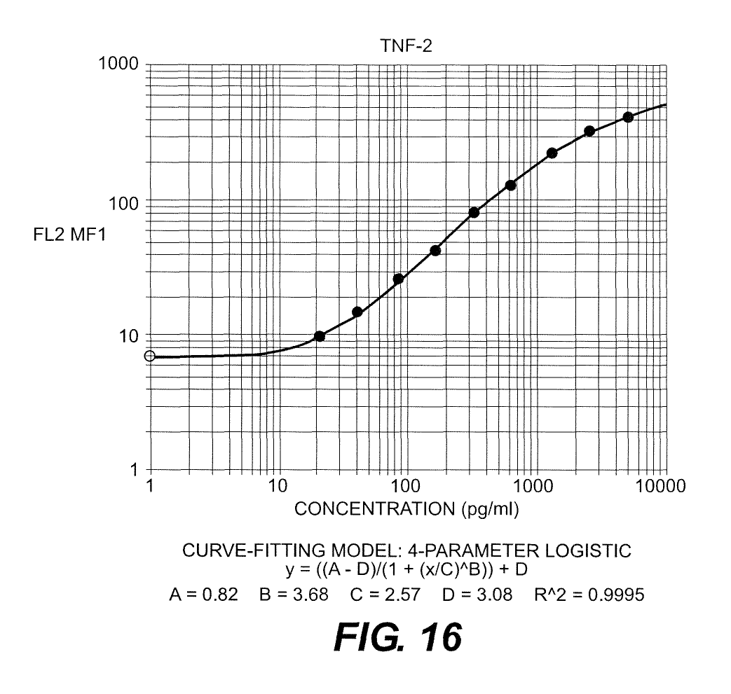

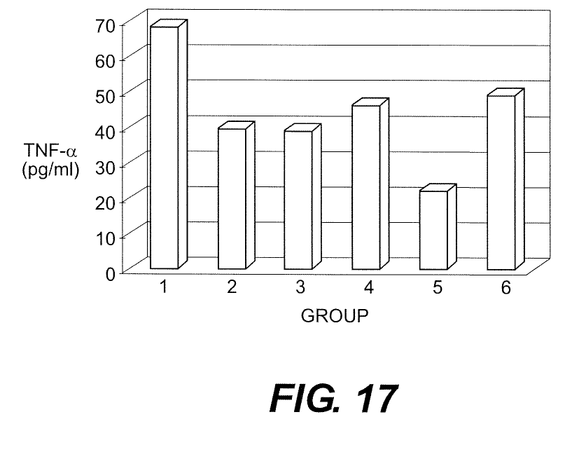

FIG. 16 shows the standard curve for flow cytometry detection of TNF-a. As shown in Table 13 and FIG. 17, serum TNF-α levels of the GA-GS high dose group was significantly higher than those of the normal control group (P<0.01). Serum TNF-a levels of the GA-GS medium and low dose groups were also statistically higher than that of the normal control group (P<0.05). The serum TNF-α levels correlated with the GS dosage. The serum TNF-α level of the virus control group was statistically higher than that of the normal control group (P<0.05). No significant difference between the PI-GS groups and the GA-GS groups was observed.

TABLE 13
Effects of GS on the blood TNF-α level of the experimental mice

| | Animals | | |
|---|---|---|---|
| Group | evaluated | TNF-α levels (x ± s) (pg/ml) | P value |
| H | 7 | 68.7 ± 49 | H versus N, <0.01 |
| M | 9 | 40.12 ± 9.5 | M versus H, <0.05 |
| L | 7 | 37.54 ± 7.65 | L versus H, <0.05 |
| C | 8 | 46.81 ± 15.49 | |
| N | 10 | 22.74 ± 7.61 | |
| P | 7 | 49.7 ± 7.59 | N versus P, <0.05 |

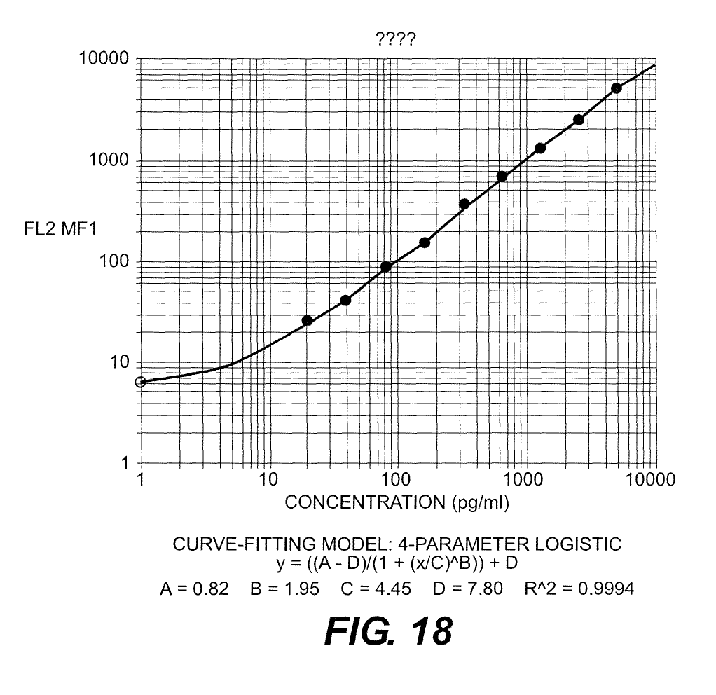

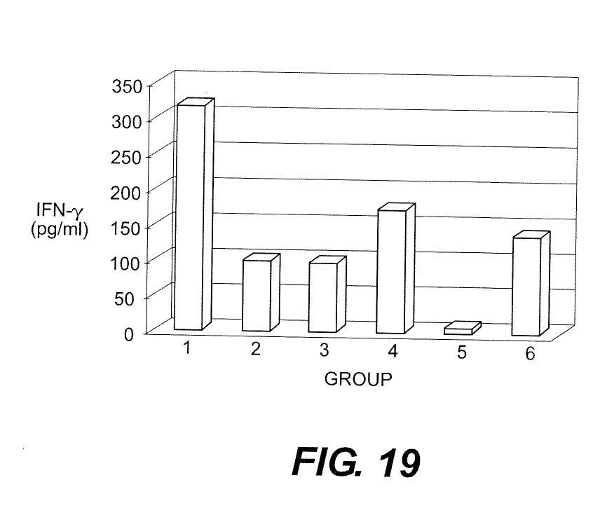

FIG. 18 shows the standard curve for flow cytometry detection of IFN-γ. As shown in Table 14 and FIG. 19, serum IFN-γ levels of each of the GA-GS group, the virus control group and the CMC control group were statistically higher than that of the normal control group (P<0.01). The serum IFN-γ levels of the GA-GS high dose group were statistically higher than those of the virus control group (P<0.05). No significant difference between the PI-GS groups and the GA-GS groups was observed.

TABLE 14
Effects of GS on serum IFN-γ levels of the experimental mice

| | Animals | | |
|---|---|---|---|
| Group | evaluated | IFN-γ levels (x ± s) (pg/ml) | P value |
| H | 7 | 306.18 ± 239.96 | H versus N, <0.01 |
| M | 9 | 101.9 ± 34.616 | M versus N, <0.01 |
| L | 7 | 88.32 ± 15.826 | L versus N, <0.01 |
| C | 8 | 174.31 ± 65.38 | C versus N, <0.01 |
| N | 10 | 6.98 ± 5.38 | |
| P | 7 | 138.64 ± 29.04 | P versus N, <0.05 |

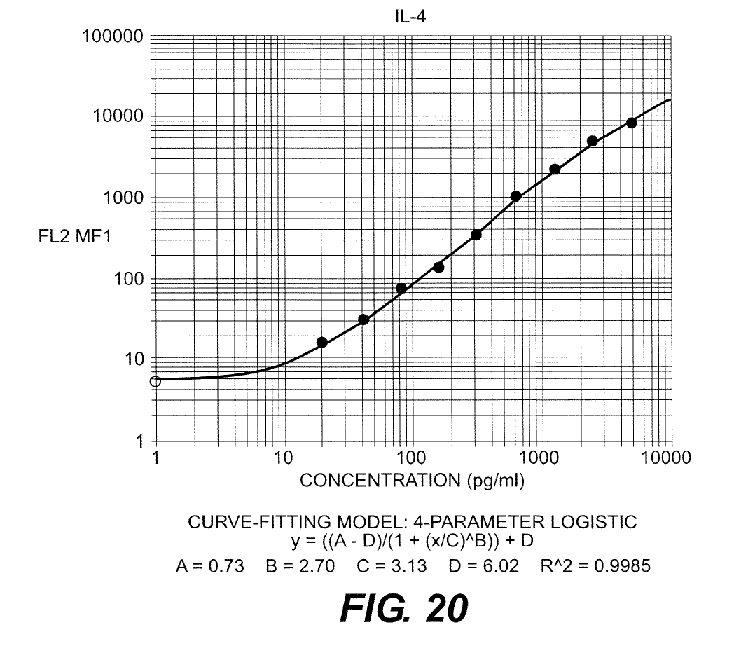

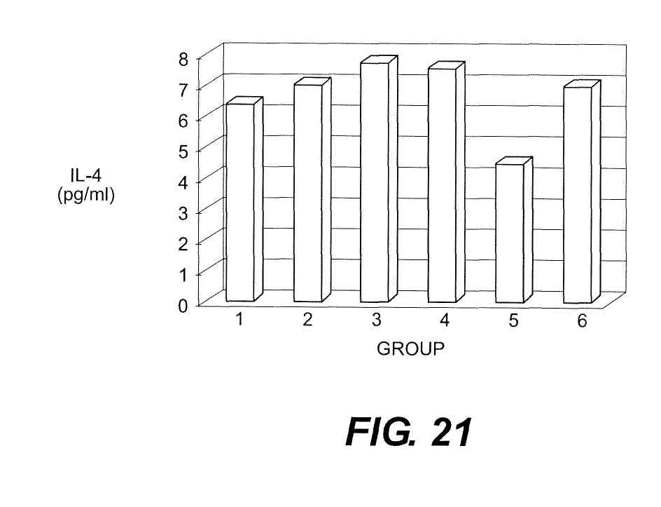

FIG. 20 shows the standard curve of flow cytometry detection of IL-4. As shown in Table 15 and FIG. 21, serum IL-4 levels of the GA-GS medium and low dose groups were statistically higher than that of the normal control group (P<0.01). Among the GA-GS treatment groups, the serum IL-4 levels appeared to be reversely correlated with the GA-GS dosage. There was no statistically significant difference between the blood IL-4 levels of the GS high dose group and the normal control group. The blood IL-4 levels of the virus control group and the CMC control group were statistically higher than that of the normal control group (P<0.05). No significant difference between the PI-GS groups and the GA-GS groups was observed.

TABLE 15
Effects of GS on serum IL-4 levels of the experimental mice

| | Animals | | |
|---|---|---|---|
| Group | evaluated | IL-4 level (x ± s) (pg/ml) | P value |
| H | 7 | 6.5 ± 1.49 | |
| M | 9 | 7.1 ± 2.19 | M versus N, <0.01 |
| L | 7 | 7.86 ± 0.86 | L versus N, <0.01 |
| C | 8 | 7.62 ± 1.89 | C versus N, <0.01 |
| N | 10 | 4.5 ± 2.76 | |
| P | 7 | 7.1 ± 0.54 | P versus N, <0.01 |

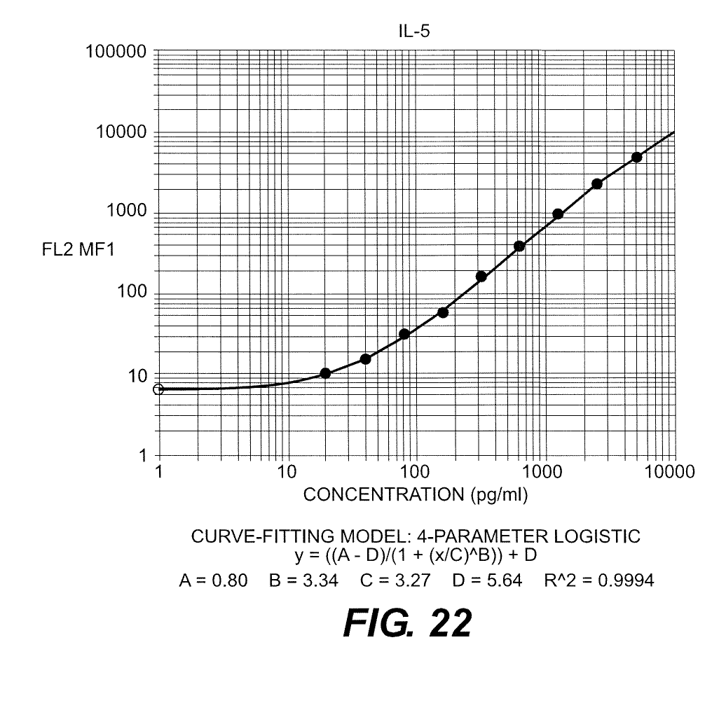

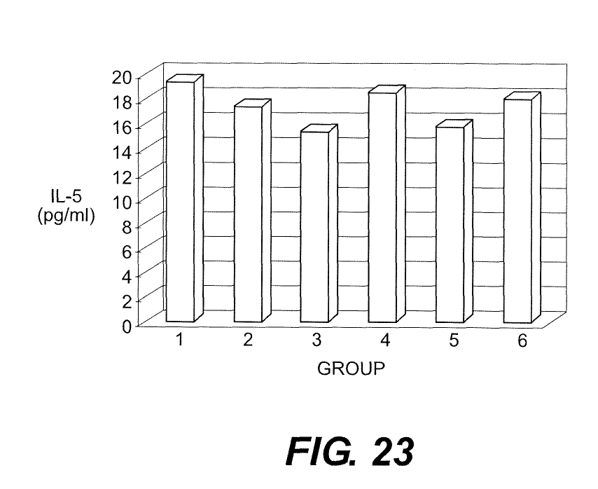

FIG. 22 shows the standard curve for flow cytometry detection of IL-5. As shown in Table 16 and FIG. 23, blood IL-5 levels were not statistically different among different experimental groups.

TABLE 16
Effects of GS on serum IL-5 levels of the experimental mice

| Group | Animals evaluated | IL-5 level (x ± s) (pg/ml) |
|---|---|---|
| H | 7 | 19.42 ± 6.15 |
| M | 9 | 17.58 ± 3.67 |
| L | 7 | 15.48 ± 1.58 |
| C | 8 | 18.74 ± 3.46 |
| N | 10 | 15.83 ± 12.22 |
| P | 7 | 18.2 ± 9.54 |

Note:
The statistical analysis demonstrated tha serum IL-5 levels among different groups were not statistically significant (P > 0.05).

### Example 8

### Detection of Viral Antigens

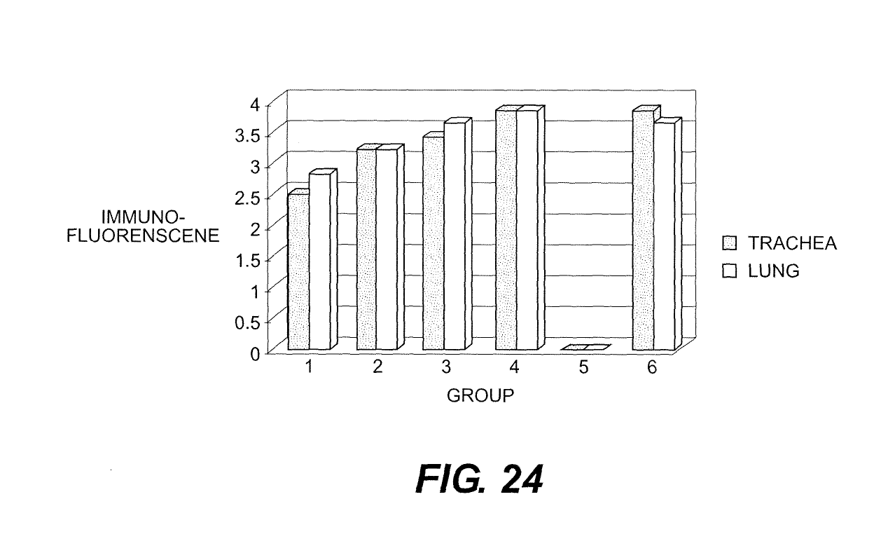

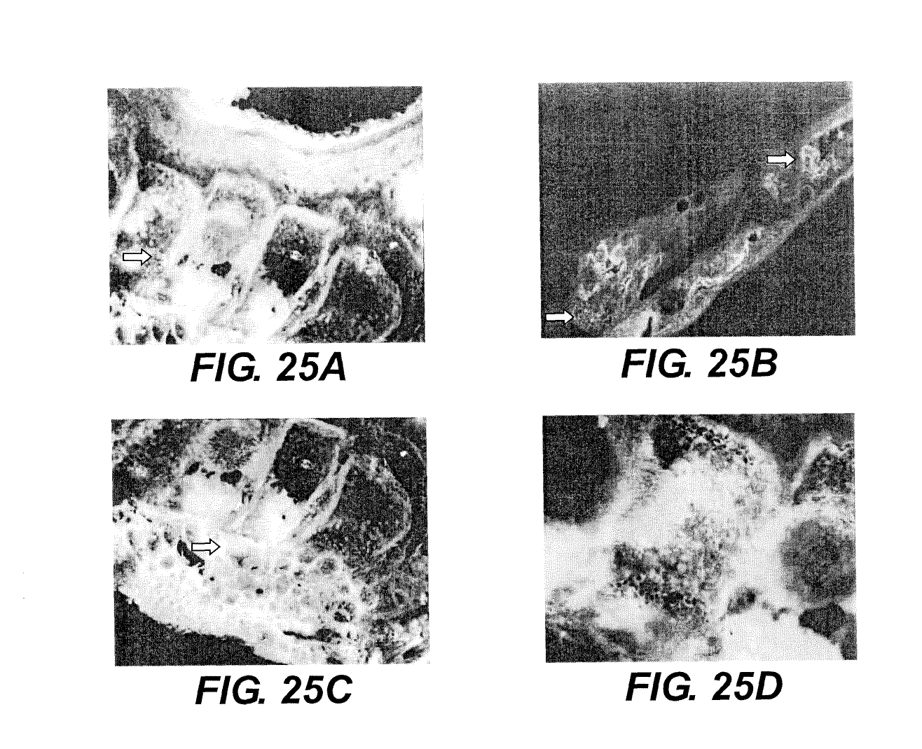

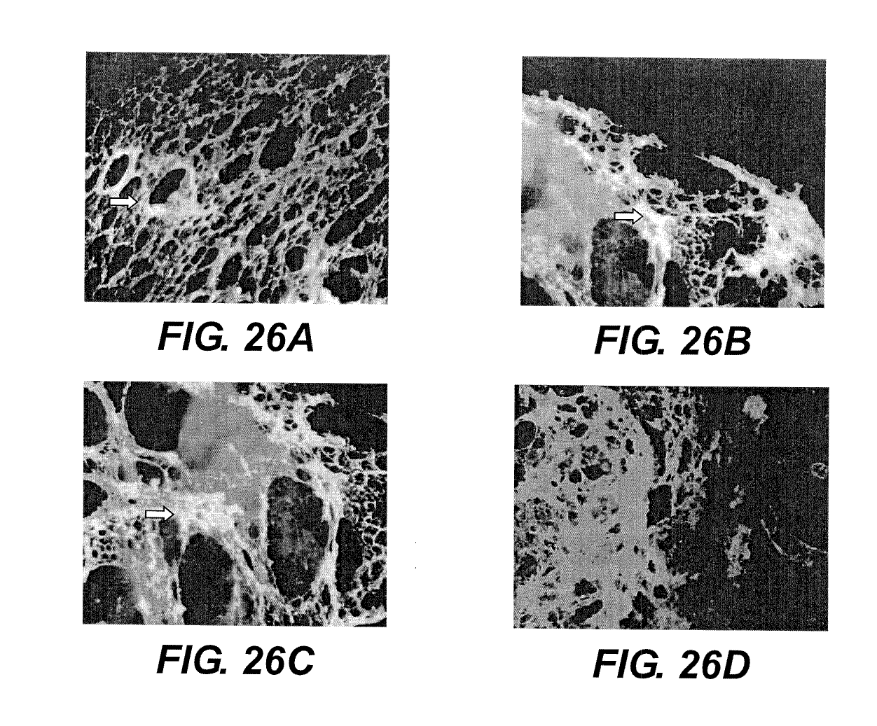

Frozen sections of lungs and trachea harvested from mice of different experimental groups were analyzed for the presence of viral antigens by indirect immumofluorescence method using chicken anti-influenza virus antiserum as the primary antibody. As shown in Table 17 and FIGS. 24-26, positive signals were detected in the cytoplasm of tissues harvested from the virus control group, the CMC control group, and all the GS dose groups, confirming the existence of the influenza virus antigen. No signals were detected in tissues from the normal control group.

TABLE 17
The immunofluorescence signal intensity from lung and trachea tissues
harvested from mice in different experimental groups

| | Animals | | | |
|---|---|---|---|---|
| Group | evaluated | Lung tissue | Trachea | P < 0.01 |
| H | 7 | ++~+++ | ++~+++ | |
| M | 9 | +++~++++ | +++~++++ | |
| L | 7 | +++~++++ | +++~++++ | |
| C | 8 | +++~++++ | +++~++++ | |
| N | 10 | — | — | H versus N, <0.01 |
| P | 7 | +++~++++ | +++~++++ | H versus P, <0.01 |

### Example 9

### Detection of Influenza Virus Hemagglutination Inhibition Antibody in the Serum of the Experimental Mice

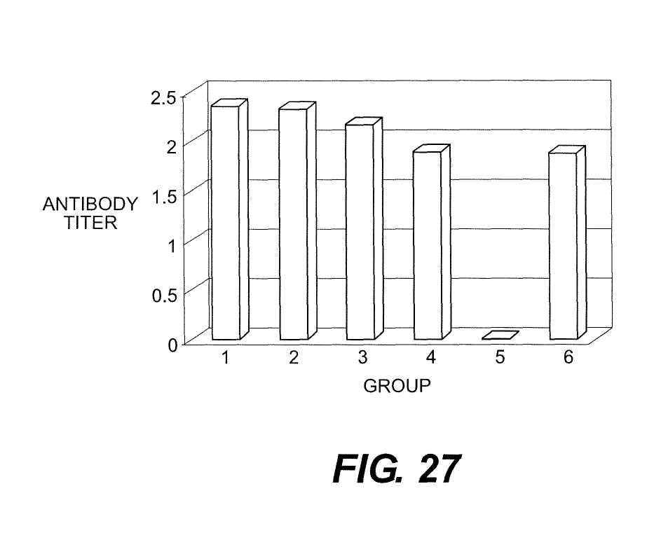

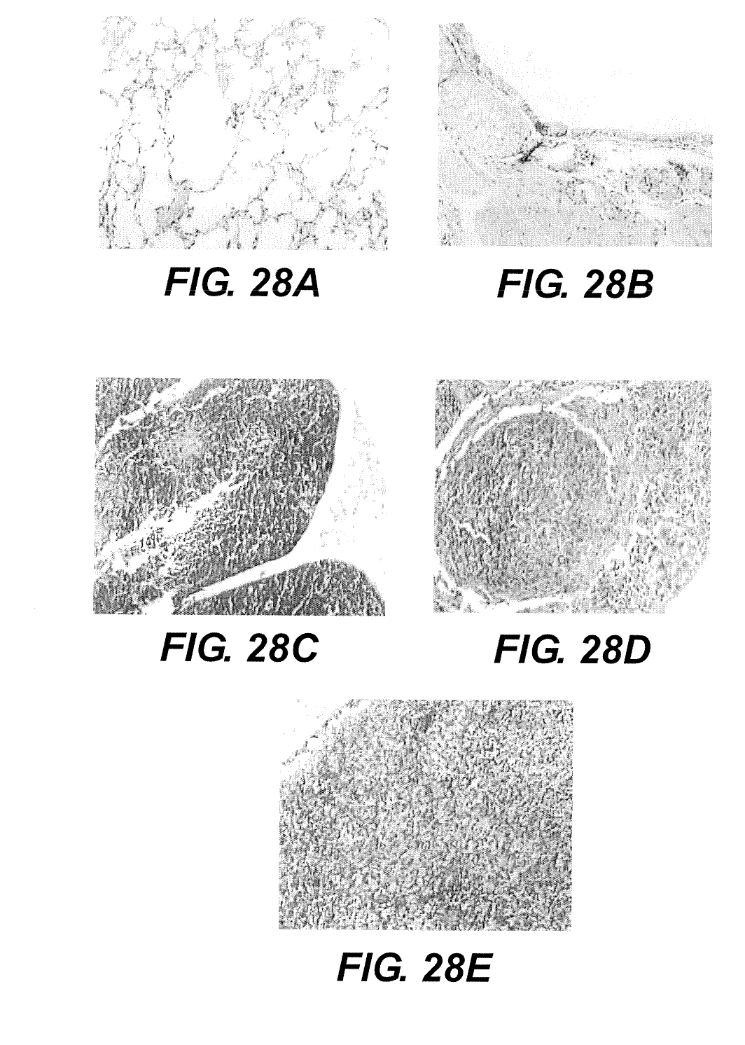

As shown in Table 18 and FIG. 27, hemagglutination inhibition antibody was detectable in the serum of the virus control group, the CMC control group, and all of the GA-GS dose groups. Serum from the normal control group was negative. In addition, the increases in antibody geometric mean titer (GMT) of the GA-GS high, medium and low dose groups were statistically higher than that of the virus control group (P<0.05). No significant difference between the PI-GS groups and the GA-GS groups was observed.

TABLE 18
Titer of hemagglutination inhibition antibody against influenza
virus in mice from different experimental groups

| | Animals | Antibody geometricr mean | |
|---|---|---|---|
| Group | evaluated | titer (x ± s) | P value |
| H | 7 | 2.38 ± 0.015 | H versus P, <0.05 |
| M | 9 | 2.35 ± 0.021 | M versus P, <0.05 |
| L | 7 | 2.2 ± 0.013 | L versus P, <0.05 |
| C | 8 | 1.9 ± 0.012 | |
| N | 10 | 0 | |
| P | 7 | 1.9 ± 0.01 | |

### Example 10

### Pathomorphological Changes in Major Organs of the Experimental Mice

Representative HE staining of tissue sections from mice in different experimental groups were shown in FIGS. 28-31. In the normal control group, structures of lung, trachea, spleen, thymus and lymph node were normal. No apparent pathological changes was observed (FIG. 28).

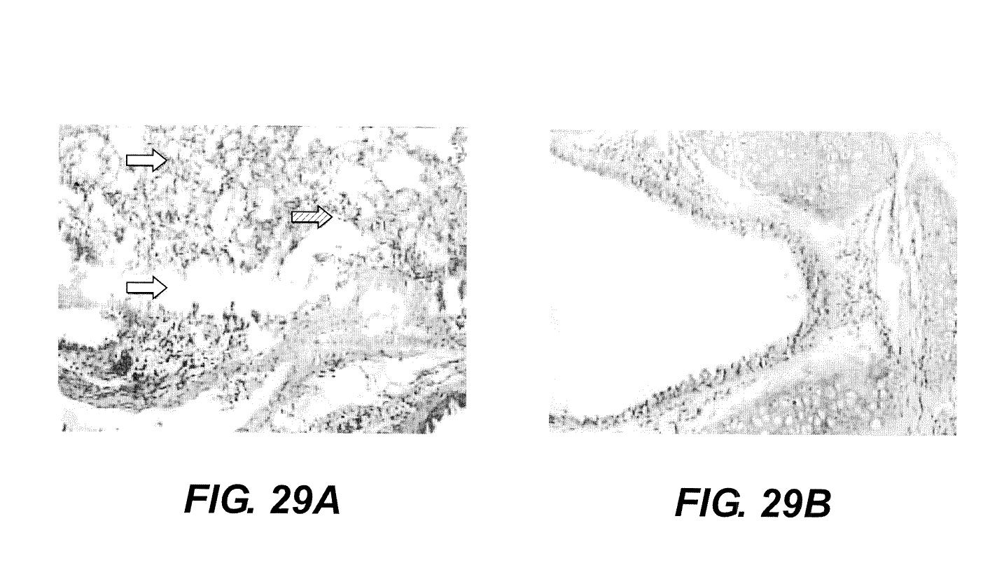

In the GA- and PI-GS low dose group, interstitial pneumonia (+++) and medium degree (++) of pulmonary alveolus soakage in alveolar cavity were observed in the lung tissue of all the mice (FIG. 29). The tissue structure of trachea, spleen, thymus and lymph node were normal, and no apparent pathological changes were observed.

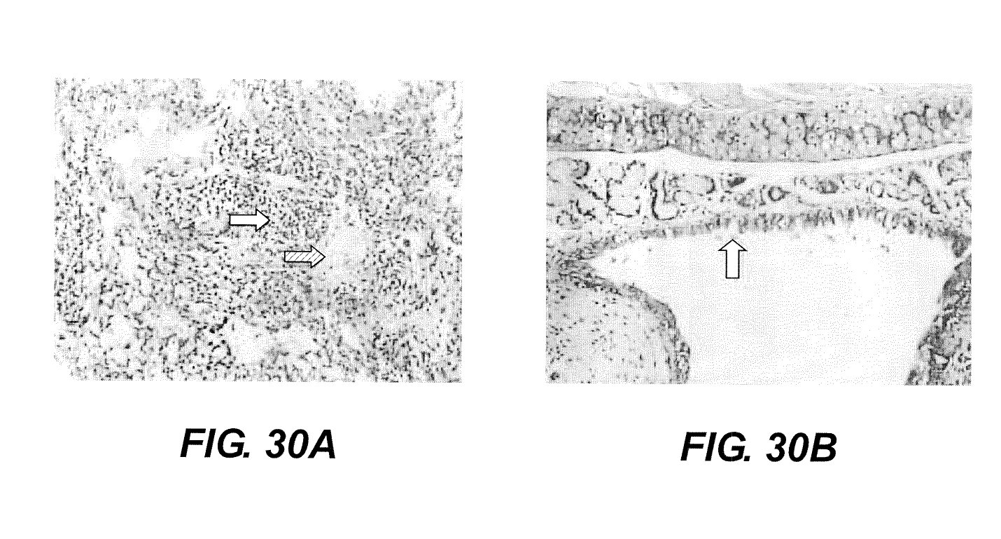

In the GA- and the PI-GS medium dose group: two fifths of the mice presented medium degree (++) of bronchopneumonia; four fifths of mice presented slight to medium degree (+˜++) of interstitial pneumonia; two fifths mice presented medium degree (++) of trachea mucous membrane degradation. Medium to severe degree (+˜+++) of pulmonary alveolus soakage in alveolar cavity were observed in all the mice from this group (FIG. 30). The structure of spleens, thymus and lymph node was normal. No apparent pathological changes were observed.

In the GA- and PI-GS high dose group: the mice presented slight degree (+) of bronchopneumonia and interstitial pneumonia; slight to medium degree (+˜++) of pulmonary alveolus soakage in alveolar cavity; slight to medium degree (+˜++) of trachea mucous membrane degradation. The structure of spleens, thymus and lymph node was normal, and no apparent pathological changes were observed.

In the CMC control group: 3/5 mice presented medium degree (++) of bronchopneumonia. 4/5 mice presented slight to medium degree (+˜++) of interstitial pneumonia. 4/5 mice presented medium degree (++) of trachea mucous membrane degradation. All the mice presented medium to severe (+˜+++) degree of pulmonary alveolus soakage in alveolar cavity. The structure of spleen, thymus and lymph node were normal. No apparent pathological changes were observed.

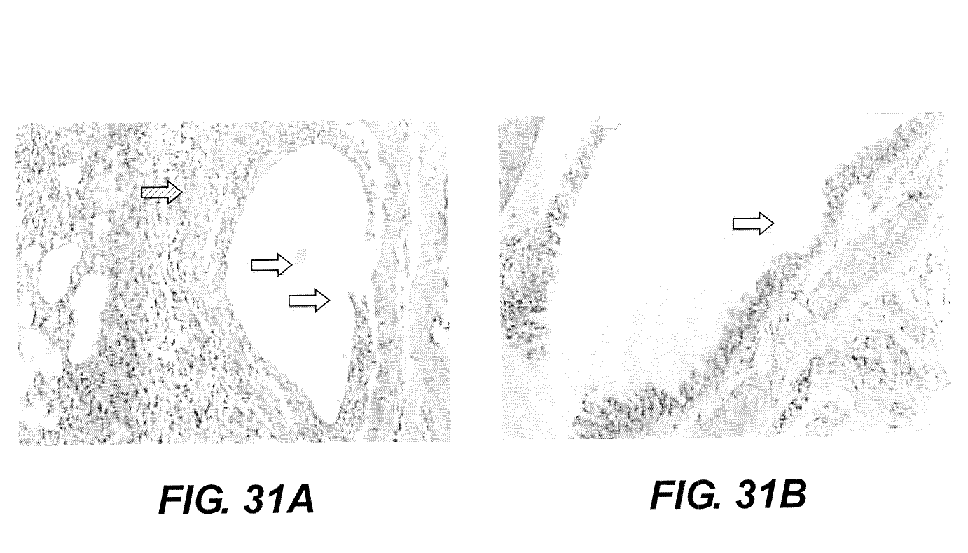

In the virus control group, all the mice presented slight to medium degree (+˜++) of interstitial pneumonia, and slight to medium degree (+˜++) of pulmonary alveolus soakage in alveolar wall. 5/6 mice presented medium degree (+˜++) of bronchopneumonia. 2/6 mice presented slight to medium degree (+˜++) of trachea mucous membrane degradation (FIG. 31). The structure of spleen, thymus and lymph node was normal. No apparent pathological changes were observed.

### CONCLUSIONS

### (1) Effects of Gs on the Thymus Index:

Thymus and spleen are important immunity organs for the body. T lymphocyte developed, differentiated and matured in thymus, and B cells inhabit mainly in the spleen. Compared with the virus control group, the three GS groups showed enhanced thymus index (P<0.05). The increase in the thymus index correlated with both the GA-GS and PI GS dosages.

### (2) Effects of Gs on Immunocytes:

Compared with the virus control group, the B cell counts in the peripheral blood of mice treated with both the GA- and PI-GS increased after viral infection. The increase correlated with the GS dosage, and was most significant in mice from the high GS dose group (P<0.05), implying that GS may have the effect of improving humoral immunity in mice. CD4+ T cell counts in the GS medium dose group, GS low dose group, virus control group and CMC control group were higher than those of the normal control group, and the differences were statistically significant (P<0.05). In addition, the NK cell counts increased in GS high and medium dose groups compared with that of the virus control group.

### (3) Effects of Gs on Cytokine Levels:

Compared with the virus control group, serum IL-2 levels increased in all of the GA- and PI-GS dose groups. The increase correlated with the GS dosage. Compared with the virus control group, serum TNF-α and IFN-γ levels increased in the GS high dose group, which would lead to increased differentiation of TH₁ cells and inhibition the differentiation of TH₂ cells, which in turn might result in specific immunological responses predominated by cellular immunity in mice infected with influenza virus.

IFN-γ, also named as immunity interferon, belongs to type II interferon and is an important cytokine with broad-spectrum antivirus effects. IFN-γ binds to the IFN-γ receptors on the cell surface and induces expression of an antiviral protein (AVP) having enzymatic activity. In the present research, serum IFN-γ levels in both the germination-activated and the pressure-induced GS high dose group were significantly higher than that of the virus control group (P<0.01). The elevated serum IFN-γ levels would be beneficial to the recovery of mice from the influenza virus infection.

### (4) Effects of Gs on the Production of Hemagglutination Inhibition Antibody:

Compared with the virus control group, the hemagglutination inhibition antibody in the blood of the three GS dose groups increased (P<0.05). The increases correlated with both the GA- and PI-GS dosage, suggesting that GS may augment humoral immunity.

### (5) Effects of Gs on the Elimination of the Viral Antigen:

Results from the immunofluorescence detection of influenza viral antigen in the lung and trachea tissue of the experimental mice indicated that the average viral antigen level in both the GA- and the PI-GS high dose group was statistically lower than that of the virus control group (P<0.05). These results suggest that GS promotes the elimination of influenza virus.

(6) Effects of Gs on the Pathomorphology of Major Organs in Mice Infected with Influenza Virus:

All mice in the virus control group presented interstitial pneumonia and bronchopneumonia, confirming that the mouse model of influenza virus has been successfully established in the present experiment. Compared with the virus control group, pathology changes in the three GS dose groups are less severe, especially in the PI-GS high dose group. These results indicate that GS has a beneficial effect on the recovery from influenza virus infection in mice.

In summary, GS is a natural material that contains various effective ingredients and has no toxic or side effect. GS is capable of modulating cellular and humoral immunity, and is effective in the prevention of influenza virus infection. Through a germination-activation process, the bioactivity of the GS is activated which provides the GS with beneficial effects on tumor inhibition and immunological enhancement. In this invention, the inventor further discovered that the “activation” process can be replaced by a pressure-induction process, and the resulting GS demonstrated better tumor inhibition and immunological enhancement effects than those produced by the germination activation process.

The embodiments illustrated and discussed in this specification are intended only to teach those skilled in the art the best way known to the inventors to make and use the invention. Nothing in this specification should be considered as limiting the scope of the present invention. The above-described embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described.

What is claimed is:

1. A method for producing pressure-induced sporoderm-broken ganoderma spores comprising:
soaking ganoderma spores in a solution to obtain soaked ganoderma spores;
drying said soaked ganoderma spores using a freeze-drying or a vacuum-drying method to obtain dried ganoderma spores;
breaking sporoderms of said dried ganoderma spores with enzyme or a mechanical method to obtain sporoderm-broken ganoderma spores;
pressurizing and depressurizing said sporoderm-broken ganoderma spores to obtain said pressure-induced sporoderm-broken ganoderma spores.

2. The method according to claim 1, where in said ganoderma spores are spores of ganoderma lucidum.

3. The method according to claim 1, wherein said ganoderma spores are selected from mature ganoderma spores.

4. The method according to claim 1, wherein said solution for soaking said ganoderma spores is water.

5. The method according to claim 1, wherein said drying method is a freeze-drying method.

6. The method according to claim 1, wherein said sporoderm-broken ganoderma spores are broken by a mechanical means.

7. The method according to claim 1, wherein said pressurizing and depressurizing process is conducted in a pressure chamber at a pressure of about 1 to 30 M Pa.

8. The method according to claim 7, wherein said pressure is about 5-10 M Pa.

9. The method according to claim 1, wherein said pressure-induced sporoderm-broken ganoderma spores are sterilized before packing into a capsule.

10. A method for ameliorating symptoms caused by influenza virus infection in a mammal, comprising:
administering to said mammal an effective amount of said pressure-induced sporoderm-broken ganoderma spores according to claim 1, wherein said mammal is susceptible to or suffering from influenza.

11. The method according to claim 10, wherein said mammal is a human.

12. The method according to claim 10, wherein said effective amount of said pressure-induced sporoderm-broken ganoderma spores is between 0.01 and 20 g/kg body weight/day.

13. The method according to claim 10, wherein said effective amount of said pressure-induced sporoderm-broken ganoderma spores is between 0.3 and 1.2 g/kg body weight/day.

14. The method according to claim 10, wherein said influenza virus is an avian influenza virus.

15. The method according to claim 1, wherein said influenza virus is a mammalian influenza virus.

16. The method according to claim 15, wherein said mammalian influenza virus is an influenza A virus.

17. The method according to claim 16, wherein said mammalian influenza virus is an FM1 strain influenza virus.

18. The method according to claim 10, wherein said said pressure-induced sporoderm-broken ganoderma spores is administered orally.

19. A method for preventing influenza in a mammal, comprising:
administering to a mammal susceptible to influenza an effective amount of said pressure-induced sporoderm-broken ganoderma spores according to claim 1 for at least seven consecutive days.

20. A method for stimulating host immune system in a mammal susceptible to or suffering from influenza virus infection, comprising:
administering to said mammal a composition comprising an effective amount of said pressure-induced sporoderm-broken ganoderma spores.

21. A pressure-induced sporoderm-broken ganoderma spore which is prepared according to claim 1.
